(19) United States
(12) Patent Application Publication
(10) Pub. No.: US 2021/0213176 A1
(43) Pub. Date: Jul. 15, 2021

(54) CHEMICAL VAPOR DEPOSITION OF POLYMER COATINGS FOR CONTROLLED DRUG RELEASE, ASSEMBLIES CONTAINING SAME, AND METHODS OF PRODUCTION AND USE THEREOF

(71) Applicant: The Board of Regents for the Oklahoma Agricultural and Mechanical Colleges, Stillwater, OK (US)

(72) Inventors: Yu MAO, Stillwater, OK (US); Bin ZHI, Xiamen (CN)

(21) Appl. No.: 17/150,246

(22) Filed: Jan. 15, 2021

Related U.S. Application Data
(60) Provisional application No. 62/961,400, filed on Jan. 15, 2020.

(51) Int. Cl. A61L 31/04; A61L 31/16; B05D 1/00

(52) U.S. Cl. CPC A61L 31/048; A61L 31/16; B05D 1/60; A61L 2300/43; A61L 2300/602; A61L 2420/06; A61L 2300/606; A61L 2420/02

(57) ABSTRACT

Drug-eluting polymer coatings for biomedical implants are disclosed, as well as assemblies and kits containing same. The polymer coatings are deposited on the biomedical implants via a solvent-free, chemical vapor deposition process. The polymer coatings exhibit controlled release of the drug with substantially no burst release and substantially linear release over time. Also disclosed are methods of making and using the coatings, assemblies, and kits.

### CROSS REFERENCE TO RELATED APPLICATIONS/INCORPORATION BY REFERENCE

This application claims benefit under 35 USC § 119(e) of provisional application U.S. Ser. No. 62/961,400, filed Jan. 15, 2020. The entire contents of the above-referenced patent application are hereby expressly incorporated herein by reference.

### STATEMENT REGARDING FEDERALLY SPONSORED RESEARCH OR DEVELOPMENT

Not Applicable.

### BACKGROUND

Coatings for use as drug delivery systems are known in the art, particularly for augmenting the capabilities of medical devices (including, but not limited to, medical implants). Drug eluting medical devices have emerged as a leading biomedical device for the treatment of various diseases and conditions. For example, in treating coronary artery diseases, drug eluting stents (DES) have been demonstrated to substantially reduce adverse events such as in-stent restenosis observed for bare metal stents in the short term by administering drug locally to suppress vascular smooth muscle proliferation.

Strategies in formulating an even and sustained drug release include tailoring the composition of the drug-embedding polymer and adding additional polymer layers as diffusion barriers. However, the processing of polymer coatings onto the surface of biomedical devices usually involves the use of solvents, which dissolve drug molecules and result in burst release at the initial stage of drug delivery from medical devices. To the best of our knowledge, strategies for zero-order drug release beyond four weeks have not been specifically identified for drug-eluting implants.

Therefore, there is a need in the art for new and improved methods for preparing drug-eluting coatings with desired controlled release levels, as well as biomedical implants coated with same. It is to such new and improved compositions, methods, assemblies, and kits that the present disclosure is directed.

### BRIEF DESCRIPTION OF THE DRAWINGS

This patent or application file contains at least one drawing executed in color. Copies of this patent or patent application publication with color drawing(s) will be provided by the Office upon request and payment of the necessary fee.

FIG. 1 schematically illustrates a chemical vapor deposition of polymer coatings in accordance with the present disclosure.

FIG. 2 shows FTIR spectra of PME, PE, and PDE coatings (Panel A), and FTIR spectra of coatings with different molar ratio of DMAEMA to EGDA (Panel B).

FIG. 3 shows PDE31 coated stent after delivery balloon expansion at 9 atm (Panel A) and close view of the coated stent struts (Panel B).

FIG. 4 shows cumulative atorvastatin release from 300-nm coatings: Panel A, PME and PDE with 50 μg/cm² atorvastatin; and Panel B, PDE31, PDE51, and PDE71 with 300 μg/cm² atorvastatin.

FIG. 5 shows drug release through PDE31 coatings: Panel A, release of atorvastatin through 300-nm coating at the dose density of 20, 50, 100, and 300 μg/cm²; Panel B, linear dependence of atorvastatin daily release rate on atorvastatin dose; Panel C, release of atorvastatin through coatings of different thickness at the dose density of 300 μg/cm²; and Panel D, release of sirolimus through 300-nm coating at the dose density of 100 and 300 μg/cm².

FIG. 6 shows Relative MTT (Panel A) and BrdU (Panel B) activity of HCASMC after two weeks of culture on PDE31 coated atorvastatin with dose density of 0, 5, 10, 100, and 300 μg/cm². Stainless steel (SS) was used as the control. Significant differences between samples were marked with stars at the level of significance given as *=0.05 and **=0.005.

FIG. 7 shows relative platelet adhesion on PDE31 nanocoated atorvastatin with various drug dose densities. Significant differences between samples were marked with stars at the level of significance given as *=0.05 and **=0.005.

FIG. 8 shows a schematic of DEX encapsulation by vapor deposition of the linear polymer of PBA or the crosslinked polymer of PEGDA.

FIG. 9 shows the release kinetics of DEX-loaded devices encapsulated by solution-coated PBA (s), vapor-deposited PBA (v), and vapor-deposited PEGDA (v). The devices had the same DEX dose density at 300 μg/cm².

FIG. 10 shows the release kinetics of PEGDA encapsulated DEX at varying dose densities (Panel A), and correlation of average DEX release rate with the dose density (Panel B).

FIG. 11 shows a daily release rate for 300 μg/cm² DEX encapsulated by PEGDA with thickness of 300 nm (Panel A), 600 nm (Panel B), and 900 nm (Panel C), and the correlation of average daily release rate with PEGDA thickness (Panel D).

FIG. 12 shows a cumulative release profile of PEGDA encapsulated DEX for 60 days (dose density of 300 μg/cm²).

FIG. 13 shows representative images of microglia with the nuclei (DAPI, blue) and the CD68 membrane protein (FITC, green) stained. Results were compared between LPS-stimulated and non-stimulated cells with and without DEX release. The LPS concentration was 100 ng/ml, and the DEX dose density was 100 μg/cm².

FIG. 14 shows a relative immunofluorescence intensity per cell for microglia incubated on PEGDA encapsulated DEX at 0, 20, 50, and 100 μg/cm² under non-stimulated (blue bar) and LPS-stimulated (orange bar) conditions on day one (left) and day three (right).

FIG. 15 shows a morphology of the microelectrode probe after 300-nm PEGDA encapsulation of 300 μg/cm² DEX (Panel A), and the impedance spectra of the microelectrode probe before and after 300-nm PEGDA encapsulation of 300 μg/cm²DEX (Panel B).

### DETAILED DESCRIPTION

Before explaining at least one embodiment of the inventive concept(s) in detail by way of exemplary language and results, it is to be understood that the inventive concept(s) is not limited in its application to the details of construction and the arrangement of the components set forth in the following description. The inventive concept(s) is capable of other embodiments or of being practiced or carried out in various ways. As such, the language used herein is intended to be given the broadest possible scope and meaning; and the embodiments are meant to be exemplary—not exhaustive. Also, it is to be understood that the phraseology and terminology employed herein is for the purpose of description and should not be regarded as limiting.

Unless otherwise defined herein, scientific and technical terms used in connection with the presently disclosed inventive concept(s) shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques are used for chemical syntheses and chemical analyses.

All patents, published patent applications, and non-patent publications mentioned in the specification are indicative of the level of skill of those skilled in the art to which this presently disclosed inventive concept(s) pertains. All patents, published patent applications, and non-patent publications referenced in any portion of this application are herein expressly incorporated by reference in their entirety to the same extent as if each individual patent or publication was specifically and individually indicated to be incorporated by reference.

All of the compositions and/or methods disclosed herein can be made and executed without undue experimentation in light of the present disclosure. While the compositions and methods of the inventive concept(s) have been described in terms of particular embodiments, it will be apparent to those of skill in the art that variations may be applied to the compositions and/or methods and in the steps or in the sequence of steps of the methods described herein without departing from the concept, spirit, and scope of the inventive concept(s). All such similar substitutions and modifications apparent to those skilled in the art are deemed to be within the spirit, scope, and concept of the inventive concept(s) as defined by the appended claims.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the term “about” is used to indicate that a value includes the inherent variation of error for a composition/apparatus/device, the method being employed to determine the value, or the variation that exists among the study subjects. For example, but not by way of limitation, when the term “about” is utilized, the designated value may vary by plus or minus twenty percent, or fifteen percent, or twelve percent, or eleven percent, or ten percent, or nine percent, or eight percent, or seven percent, or six percent, or five percent, or four percent, or three percent, or two percent, or one percent from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “substantially pure” means an object species is the predominant species present (i.e., on a molar basis it is more abundant than any other individual species in the composition), and preferably a substantially purified fraction is a composition wherein the object species comprises at least about 50 percent (on a molar basis) of all macromolecular species present. Generally, a substantially pure composition will comprise more than about 80 percent of all macromolecular species present in the composition, more preferably more than about 85%, 90%, 95%, and 99%. Most preferably, the object species is purified to essential homogeneity (contaminant species cannot be detected in the composition by conventional detection methods) wherein the composition consists essentially of a single macromolecular species.

The term “pharmaceutically acceptable” refers to compounds and compositions which are suitable for administration to humans and/or animals without undue adverse side effects such as (but not limited to) toxicity, irritation, and/or allergic response commensurate with a reasonable benefit/risk ratio.

The term “pharmaceutically-acceptable excipient” refers to any carrier, vehicle, and/or diluent known in the art or otherwise contemplated herein that may improve solubility, deliverability, dispersion, stability, and/or conformational integrity of the compositions disclosed herein.

The term “patient” as used herein includes human and veterinary subjects. “Mammal” for purposes of treatment refers to any animal classified as a mammal, including (but not limited to) humans, domestic and farm animals, nonhuman primates, and any other animal that has mammary tissue.

The term “treatment” refers to both therapeutic treatment and prophylactic or preventative measures. Those in need of treatment include, but are not limited to, individuals already having a particular condition/disease/infection as well as individuals who are at risk of acquiring a particular condition/disease/infection (e.g., those needing prophylactic/preventative measures). The term “treating” refers to administering an agent to a patient for therapeutic and/or prophylactic/preventative purposes.

A “therapeutic composition” or “pharmaceutical composition” refers to an agent that may be administered in vivo to bring about a therapeutic and/or prophylactic/preventative effect.

Administering a therapeutically effective amount or prophylactically effective amount is intended to provide a therapeutic benefit in the treatment, prevention, and/or management of a disease, condition, and/or infection. The specific amount that is therapeutically effective can be readily determined by the ordinary medical practitioner, and can vary depending on factors known in the art, such as (but not limited to) the type of condition/disease/infection, the patient's history and age, the stage of the condition/disease/infection, and the co-administration of other agents.

The term “effective amount” refers to an amount of a biologically active molecule or conjugate or derivative thereof sufficient to exhibit a detectable therapeutic effect without undue adverse side effects (such as (but not limited to) toxicity, irritation, and allergic response) commensurate with a reasonable benefit/risk ratio when used in the manner of the inventive concept(s). The therapeutic effect may include, for example but not by way of limitation, preventing, inhibiting, or reducing the occurrence of pulmonary fibrosis. The effective amount for a subject will depend upon the type of subject, the subject's size and health, the nature and severity of the condition/disease/infection to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

As used herein, the term “concurrent therapy” is used interchangeably with the terms “combination therapy” and “adjunct therapy,” and will be understood to mean that the patient in need of treatment is treated or given another drug for the condition/disease/infection in conjunction with the pharmaceutical compositions of the present disclosure. This concurrent therapy can be sequential therapy, where the patient is treated first with one pharmaceutical composition and then the other pharmaceutical composition, or the two pharmaceutical compositions are given simultaneously.

The terms “administration” and “administering,” as used herein, will be understood to include all routes of administration known in the art, including but not limited to, oral, topical, transdermal, parenteral, subcutaneous, intranasal, mucosal, intramuscular, intraperitoneal, intravitreal, and intravenous routes, and including both local and systemic applications. In addition, the compositions of the present disclosure (and/or the methods of administration of same) may be designed to provide delayed, controlled, or sustained release using formulation techniques which are well known in the art.

Turning now to the inventive concept(s), certain non-limiting embodiments thereof are directed to polymer coatings for the encapsulation of drugs as well assemblies containing same and methods of producing and using same.

Certain non-limiting embodiments of the present disclosure are directed to an assembly that includes: a biomedical implant; a polymer coating deposited on at least a portion of at least one surface of the biomedical implant; and at least one drug disposed within the coating. The assembly exhibits controlled release of the at least one drug via elution from the polymer coating.

The biomedical implant may be any device for implantation within a patient and for which elution of a drug therefrom may be desired. For example (but not by way of limitation), the biomedical implant may be an arterial stent. An arterial stent comprises a tube formed of a metal mesh and/or a bioresorbable material. Other non-limiting examples of biomedical implants that can be utilized in accordance with the present disclosure are neural probes, orthopedic devices, vascular prosthetic devices, endoprosthetic devices, cardiac pacemakers, implanted cardiac defibrillators, intraocular lenses, intrauterine devices, breast implants, tympanostomy tubes, combinations thereof, and the like.

The biomedical implant may be formed of any material that allows for implantation into a patient and onto which a polymer coating can be deposited. Non-limiting examples of materials that can be utilized in accordance with the present disclosure include a metal or alloy (such as, but not limited to, stainless steel, cobalt-chrome alloy, titanium, nickel-titanium alloy (nitinol), gold, platinum, silver, iridium, niobium, tantalum, tungsten, and the like); a ceramic (such as, but not limited to, alumina, zirconia, hydroxyapatite, bioglass, silicon, carbon, and the like); a polymer (such as, but not limited to, polyethylene, polyurethane, polyamide, polymethylmethacrylate, polytetrafluoroethylene, polyurethane, and the like); and various combinations thereof.

Any drug(s) know in the art or otherwise contemplated herein may be utilized in accordance with the present disclosure, so long as elution thereof from a biomedical implant is desired. For example (but not by way of limitation), the drug may comprise an anti-proliferative drug, an anti-inflammatory drug, an antimicrobial drug, a cancer-therapy drug, a protein therapeutic, a hormone, combinations thereof, and the like. Non-limiting examples of particular drugs that may be utilized in accordance with the present disclosure include sirolimus, everolimus, zotarolimus, umirolimus, novolimus, amphilimus, atorvastatin, paclitaxel, dexamethasone, and combinations thereof.

The drug(s) and the polymer coating may be disposed upon the biomedical implant by any methods known in the art or otherwise contemplated herein. In certain non-limiting embodiments, the polymer coating is deposited upon at least a portion of at least one surface of the biomedical implant via a solvent-free, chemical vapor deposition (CVD) process. In a particular (but non-limiting) embodiment, the polymer coating is deposited upon at least a portion of at least one surface of the biomedical implant via a single-step, solvent-free, chemical vapor deposition process.

The polymer coating may be formed of one or more polymers, and the polymers may be any polymers known in the art or otherwise contemplated herein that are capable of forming a coating on a biomedical implant that will elute drug(s) via a controlled release mechanism. Non-limiting examples of polymers that may be utilized in accordance with the present disclosure include poly(2-dimethylamino ethyl methacrylate-co-ethylene glycol diacrylate) (PDE), poly(methacrylic acid-co-ethylene glycol diacrylate) (PME), poly(ethylene glycol dimethacrylate) (PEGDA), poly(2-dimethylamino ethyl methacrylate-co-mathacrylic acid-co-ethylene glycol dimethacrylate) (PDME), poly(butyl acrylate) (PBA), poly(1H,1H,2H,2H-perfluorodecyl acrylate) (PPFDA), poly(divinylbenzene) (PDVB), polytetrafluoroethylene (PTFE), and combinations thereof.

In a particular (but non-limiting) embodiment, the polymer coating comprises (P(DMAEMA)-co-EGDA). The DMAEMA may be present at about 45 mol %.

In a particular (but non-limiting) embodiment, the polymer coating comprises PDE, and the at least one drug comprises atorvastatin.

In a particular (but non-limiting) embodiment, the polymer coating comprises at least one of PBA and PEGDA.

The polymer coating may be deposited upon the biomedical implant at any thickness that will allow the assembly to function as described herein and that will allow for elution of the drug at a controlled rate as described herein. In certain non-limiting embodiments, the polymer coating has a thickness of about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, about 1.0 micron, about 1.25 micron, about 1.5 micron, about 1.75 micron, about 2.0 micron, about 2.25 micron, about 2.5 micron, about 2.75 micron, about 3.0 micron, about 3.25 micron, about 3,5 micron, about 3.75 micron, about 4.0 micron, about 4.25 micron, about 4.5 micron, about 4.75 micron, about 5.0 micron, about 5.25 micron, about 5.5 micron, about 5.75 micron, about 6.0 micron, about 6.25 micron, about 6.5 micron, about 6.75 micron, about 7.0 micron, about 7.25 micron, about 7.5 micron, about 7.75 micron, about 8.0 micron, about 8.25 micron, about 8.5 micron, about 8.75 micron, about 9.0 micron, about 9.25 micron, about 9.5 micron, about 9.75 micron, about 10 micron, or higher, as well as any range between two of the above values (such as, but not limited to, a range of from about 100 nm to about 10 micron, a range of from about 100 nm to about 1.0 micron, a range of from about 200 nm to about 1.0 micron, a range of from about 200 nm to about 500 nm, a range of from about 250 nm to about 350nm, etc.). In a particular (but non-limiting) embodiment, the polymer coating has a thickness of about 300 nm.

In certain non-limiting embodiments, the polymer coating is a polymer nanocoating. The term “nanocoating” as used herein refers to a coating that has a thickness of less than about 300 nm, such as, but not limited to, less than about 250 nm, less than about 200 nm, less than about 150 nm, less than about 100 nm, or the like.

The drug may be present in the coating at any concentration (also referred to as “dose density”) that allows the assembly to function as described herein. In certain non-limiting embodiments, the at least one drug is present in the coating in a concentration of about 1 μg/cm², about 2 μg/cm², about 3 μg/cm², about 4 μg/cm², about 5 μg/cm², about 6 μg/cm², about 7 μg/cm², about 8 μg/cm², about 9 μg/cm², about 10 μg/cm², about 20 μg/cm², about 30 μg/cm², about 40 μg/cm², about 50 μg/cm², about 60 μg/cm², about 70 μg/cm², about 80 μg/cm², about 90 μg/cm², about 100 μg/cm², about 125 μg/cm², about 150 μg/cm², about 175 μg/cm², about 200 μg/cm², about 225 μg/cm², about 250 μg/cm², about 275 μg/cm², about 300 μg/cm², about 325 μg/cm², about 350 μg/cm², about 375 μg/cm², about 400 μg/cm², about 425 μg/cm², about 450 μg/cm², about 475 μg/cm², about 500 μg/cm², 525 μg/cm², about 550 μg/cm², about 575 μg/cm², about 600 μg/cm², about 625 μg/cm², about 650 μg/cm², about 675 μg/cm², about 700 μg/cm², about 725 μg/cm², about 750 μg/cm², about 775 μg/cm², about 800 μg/cm², about 825 μg/cm², about 850 μg/cm², about 875 μg/cm², about 900 μg/cm², about 925 μg/cm², about 950 μg/cm², about 975 μg/cm², about 1 mg/cm², about 2 mg/cm², about 3 mg/cm², about 4 mg/cm², about 5 mg/cm², about 6 mg/cm², about 7 mg/cm², about 8 mg/cm², about 9 mg/cm², about 10 mg/cm², about 15 mg/cm², about 20 mg/cm², about 25 mg/cm², about 30 mg/cm², about 35 mg/cm², about 40 mg/cm², about 45 mg/cm², about 50 mg/cm², about 55 mg/cm², about 60 mg/cm², about 65 mg/cm², about 70 mg/cm², about 75 mg/cm², about 80 mg/cm², about 85 mg/cm², about 90 mg/cm², about 95 mg/cm², about 100 mg/cm², about 125 mg/cm², about 150 mg/cm², about 175 mg/cm², about 200 mg/cm², about 225 mg/cm², about 250 mg/cm², about 275 mg/cm², about 300 mg/cm², about 325 mg/cm², about 350 mg/cm², about 375 mg/cm², about 400 mg/cm², about 425 mg/cm², about 450 mg/cm², about 475 mg/cm², about 500 mg/cm², or higher, as well as a range in between any of the above values (i.e., a range of from about 1 μg/cm² to about 500 mg/cm², a range of from about 10 μg/cm² to about 500 mg/cm², a range of from about 1 μg/cm² to about 1 mg/cm², a range of from about 10 μg/cm² to about 500 μg/cm², a range of from about 50 μg/cm² to about 300 μg/cm², a range of from about 10 μg/cm² to about 300 μg/cm², etc.).

The assembly may be provided with any average daily drug release rate that allows the assembly to function in accordance with the present disclosure. For example (but not by way of limitation), the assembly may have an average daily drug release rate of about 0.1 μg/day, about 0.2 μg/day, about 0.3 μg/day, about 0.4 μg/day, about 0.5 μg/day, about 0.6 μg/day, about 0.7 μg/day, about 0.8 μg/day, about 0.9 μg/day, about 1 μg/day, about 2 μg/day, about 3 μg/day, about 4 μg/day, about 5 μg/day, about 6 μg/day, about 7 μg/day, about 8 μg/day, about 9 μg/day, about 10 μg/day, about 11 μg/day, about 12 μg/day, about 13 μg/day, about 14, μg/day, about 15 μg/day, about 20 μg/day, about 25 μg/day, about 30 μg/day, about 35 μg/day, about 40 μg/day, about 45 μg/day, about 50 μg/day, about 55 μg/day, about 60 μg/day, about 65 μg/day, about 70 μg/day, about 75 μg/day, about 80 μg/day, about 85 μg/day, about 90 μg/day, about 95 μg/day, about 100 μg/day, about 150 μg/day, about 200 μg/day, about 250 μg/day, about 300 μg/day, about 350 μg/day, about 400 μg/day, about 450 μg/day, about 500 μg/day, about 550 μg/day, about 600 μg/day, about 650 μg/day, about 700 μg/day, about 750 μg/day, about 800 μg/day, about 850 μg/day, about 900 μg/day, about 950 μg/day, about 1 mg/day, about 2 mg/day, about 3 mg/day, about 4 mg/day, about 5 mg/day, or higher, as well as a range in between two of the above values (i.e., a range of from about 0.1 μg/day to about 5 mg/day, a range of from about 1 μg/day to about 1 mg/day, a range of from about 0.1 μg/day to about 100 μg/day, a range of from about 0.2 μg/day to about 50 μg/day, a range of from about 0.5 μg/day to about 200 μg/day, a range of from about 3 μg/day to about 13 μg/day, etc.).

The assembly may exhibit controlled drug release for any desired period that allows the assembly to function in accordance with the present disclosure. For example (but not by way of limitation), the assembly may exhibit controlled drug release for a period of at least about one week, such as (but not limited to), at least about 2 weeks, at least about 3 weeks, at least about 4 weeks, at least about 5 weeks, at least about 6 weeks, at least about 8 weeks, at least about 12 weeks, at least about 16 weeks, at least about 20 weeks, at least about 25 weeks, at least about 30 weeks, at least about 35 weeks, at least about 40 weeks, at least about 45 weeks, at least about 50 weeks, or more, or a range formed of two of the above values (i.e., a range of from about one week to about 50 weeks, a range of from about 2 weeks to about 25 weeks, a range of from about 2 weeks to about 6 weeks, a range of from about 2 weeks to about 4 weeks, a range of from about 4 weeks to about 16 weeks, etc.).

In certain non-limiting embodiments, the assembly exhibits a substantially close-to-linear release of drug with substantially no burst release of drug.

In certain non-limiting embodiments, the assembly is substantially biocompatible with at least one of human endothelial cells, vascular smooth muscle cells, and platelets.

Certain non-limiting embodiments of the present disclosure are directed to a kit that includes one or more of any of the assemblies disclosed or otherwise contemplated herein. In addition to the assembly(ies), the kit may further contain other component(s) for performing any of the particular methods described or otherwise contemplated herein. For example (but not by way of limitation), the kits may additionally include other components involved in the placement of the biomedical implant and/or treatment of the patient. The nature of these additional component(s)/reagent(s) will depend upon the particular treatment format and/or area/organ to be treated, and identification thereof is well within the skill of one of ordinary skill in the art; therefore, no further description thereof is deemed necessary. In addition, the kit can further include a set of written instructions explaining how to use one or more components of the kit. A kit of this nature can be used in any of the methods described or otherwise contemplated herein.

Certain non-limiting embodiments of the present disclosure include a method of producing an assembly comprising a drug-eluting biomedical implant, such as (but not limited to), any of the assemblies described herein above or otherwise contemplated herein. The method includes the steps of: disposing at least one drug on at least a portion of at least one surface of a biomedical implant to form at least one drug layer on the biomedical implant; and depositing at least one polymer coating on the drug layer of the biomedical implant by a solvent-free, chemical vapor deposition process. As such, the at least one drug is released via a controlled manner from the polymer coating.

In a particular (but non-limiting) embodiment, the at least one polymer coating is deposited on the drug layer of the biomedical implant by a single-step, solvent-free, chemical vapor deposition process. In alternative embodiments, the deposition process involves multiple steps.

The polymer coating deposition process begins with the decomposition of an initiator in the vapor phase and the subsequent free radical polymerization of monomers on the drug layer of the biomedical implant. Suitable initiators for use in this method include, but are not limited to, tert-butyl peroxide, tert-butyl peroxybenzoate, and tert-amyl peroxide.

Any monomers known in the art or otherwise contemplated herein that can be utilized to produce a coating on a medical implant may be utilized in accordance with the present disclosure. Non-limiting examples of monomers that fall within the scope of the present disclosure include 2-dimethylamino ethyl methacrylate, methacrylic acid, hydroxyethyl methacrylate, 2-vinylpyridine, isocyanatoethyl methacrylate, 3-(trimethoxysilyl)propyl methacrylate, 1H,1H,2H,2H-perfluorodecyl acrylate, divinylbenzene, ethylene glycol diacrylate, allyl methacrylate, and the like.

Any crosslinkers known in the art or otherwise contemplated herein that can be utilized to produce a polymer coating on a medical implant may be utilized in accordance with the present disclosure. Non-limiting examples of crosslinker that fall within the scope of the present disclosure include divinylbenzene, ethylene glycol diacrylate, 3-(trimethoxysilyl)propyl methacrylate, and the like.

The selected initiator, monomer, and the optional crosslinker are preheated at a temperature suitable for vaporization of the chemicals and metered into the CVD reactor. The feeding of the initiator, the monomer, and the optional crosslinker into the CVD reactor may be achieved using a metering device such as but not limited to a mass flow controller with or without a carrier gas. The vapor flow ratio of monomer and crosslinker may be varied to provide a desired level of crosslinking. For example (but not by way of limitation), the vapor flow ratio of monomer and crosslinker may be about 0.5:1, about 1:1, about 2:1, about 3:1, about 4:1, about 5:1, about 6:1, about 7:1, about 8:1, about 9:1, about 10:1, about 11:1, about 12:1, about 13:1, about 14:1, about 15:1, or the like, as well as a range formed of two of the above values. In a particular (but non-limiting) embodiment, the vapor flow ratio of monomer and crosslinker is in a range of from about 2:1 to about 8:1.

The pressure within the reactor is maintained at a pre-determined value using a regulating device such as but not limited to a throttling butterfly valve. The CVD reactor is equipped with a device that decompose the initiator. Such a device may be but is not limited to a heated filament array operating at a temperature to decompose the initiator. CVD polymerization begins with the decomposing of the initiator followed by the reaction of the monomer with the decomposed initiator to form polymer coatings on the drug layer of the biomedical implant. The stage supporting the biomedical implant is maintained at the desired temperature by a water-circulating system associated with the stage. The water circulation is to maintain the biomedical implant at a temperature between about 5° C. and about 70° C. during the CVD process. The temperature of the biomedical implant is selected to retain the activity of the drug while promoting the deposition of the polymer coating.

The polymer coating deposition of the methods of the present disclosure may occur at any temperature and under any conditions that allow for the production of the assemblies of the present disclosure. In certain non-limiting embodiments, the chemical vapor deposition process occurs at a temperature of 5° C., about 10° C., about 15° C., about 20° C., about 25° C., about 30° C., about 35° C., about 40° C., about 45° C., about 50° C., about 55° C., about 60° C., about 65° C., about 70° C., and the like, as well as a range formed of two of the above values (i.e., a range of from about 5° C. to about 60° C., etc.).

Certain non-limiting embodiments of the present disclosure include a method that comprises the step of placing any of the assemblies described or otherwise contemplated herein within a patient in need of such treatment.

The assembly may be placed within any portion of the patient for which treatment is desired. Selection of the implantation position can be determined based upon the type of biomedical implant present in the assembly.

In a particular (but non-limiting) embodiment, the biomedical implant of the assembly is an arterial stent, and the assembly is placed within an artery of the patient.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and are meant to be exemplary, not exhaustive.

### Example 1

In this Example, a novel coating approach for sustained, zero-order release of anti-proliferative drugs was investigated. The coatings were synthesized using a single-step, solvent-free, CVD process that enables crosslinking of coatings directly on the drug layer and the in situ tailoring of coating compositions as well as the thickness thereof. Atorvastatin was chosen as the model drug because of the specific inhibition against proliferation and viability of smooth muscle cells (SMC) and additional cardioprotective effects. The atorvastatin release kinetics was systematically studied to understand the effects of coating parameters on drug release. The release kinetics of nanocoated sirolimus, an anti-proliferative drug in clinical use, were also evaluated for comparison. The impact of linearly released atorvastatin on SMC viability and proliferation were quantitatively studied, and the hemocompatibility of nanocoated atorvastatin was assessed.

### Materials and Methods

Preparation of Devices. Stainless steel slides (316L, Online Metals, Grand Prairie, Tex.) at the dimension of 0.5 cm×1.0 cm were prewashed with soap water and then with acetone (99.5%, Fisher Scientific, Hampton, N.H.) in an ultrasonic bath for 30 min each. After removal of trace acetone with ethanol (200 proof, Pharmco-Aaper, Toronto, Ontario, Canada), samples were rinsed with deionized water for three times and air dried. Atorvastatin calcium (Astatech Inc., Bristol, Pa.) was dissolved in tetrahydrofuran (Fisher Scientific, Hampton, N.H.) at 30 mg/ml, dipped on stainless steel slides or meshes at −20° C., and dried overnight. Sirolimus (LC Laboratory, Woburn, Mass.) was dissolved in methanol (Fisher Scientific, Hampton, N.H.) at 20 mg/ml, dipped on stainless steel slides or meshes at −20° C., and dried overnight. INTEGRITY® bare metal stents mounted on delivery balloons were purchased from Medtronic (Minneapolis, Minn.) and used as received.

Coating of Devices. The nanocoating process was conducted using a CVD technique described in previous studies (Ye et al., 2011; Ye et al., 2012; and Ye et al., 2017). Poly(methacrylic acid-co-ethylene glycol diacrylate) (PME) and poly(2-dimethylamino ethyl methacrylate-co-ethylene glycol diacrylate) (PDE) nanocoatings were directly polymerized on the drug-loaded devices. Di-tert-butyl peroxide (TBP) (98%, Sigma-Aldrich, St. Louis, Mo.) was used as the initiator and metered into the reactor using a 1479A mass flow controller (MKS, Andover, Mass.). The TBP flow rate was maintained at 0.16 sccm in all the depositions. The monomers of 2-dimethylamino ethyl methacrylate (DMAEMA, 98%) and methacrylic acid (MAA, 99%) and the crosslinker of ethylene glycol diacrylate (EGDA, 90%) were purchased from Sigma-Aldrich (St. Louis, Mo.). DMAEMA, MAA, and EGDA were vaporized at 50° C., 50° C., and 55° C., respectively. The flow rate of DMAEMA was controlled using a 1153 mass flow controller (MKS, Andover, Mass.), and the flow rates of MAA and EGDA were controlled using separate Swagelok needle valves. The flow rate of EGDA was set at 0.19 sccm, while the flow rate was maintained at 0.57 sccm for MAA and DMAEMA in the synthesis of PME and PDE, respectively. For PDE31, PDE51, and PDE71 nanocoatings, the flow rate of DMAEMA was set at 0.57 sccm, while the vapor flow ratio of DMAEMA to EGDA was controlled at 3:1, 5:1, and 7:1, respectively.

A parallel array of nichrome filament (Ni80/Cr20, Goodfellow, Coraopolis, Pa.), was resistively heated to 220° C. to decompose the TBP initiator. To prevent drug degradation, stainless steel devices were water-cooled to maintain the temperature at 34° C. Temperature was measured using thermocouples (Omega, Type K, Stamford, Conn.) directly mounted on the filament and the stage. A throttling butterfly valve (253B, MKS, Andover, Mass.) was used to maintain the reactor pressure at 200 mTorr. The nanocoating growth process was monitored by measuring the increase of nanocoating thickness in situ on a reference silicon wafer using interferometry equipped with a 633 nm He—Ne laser (JDS Uniphase, Milpitas, Calif.).

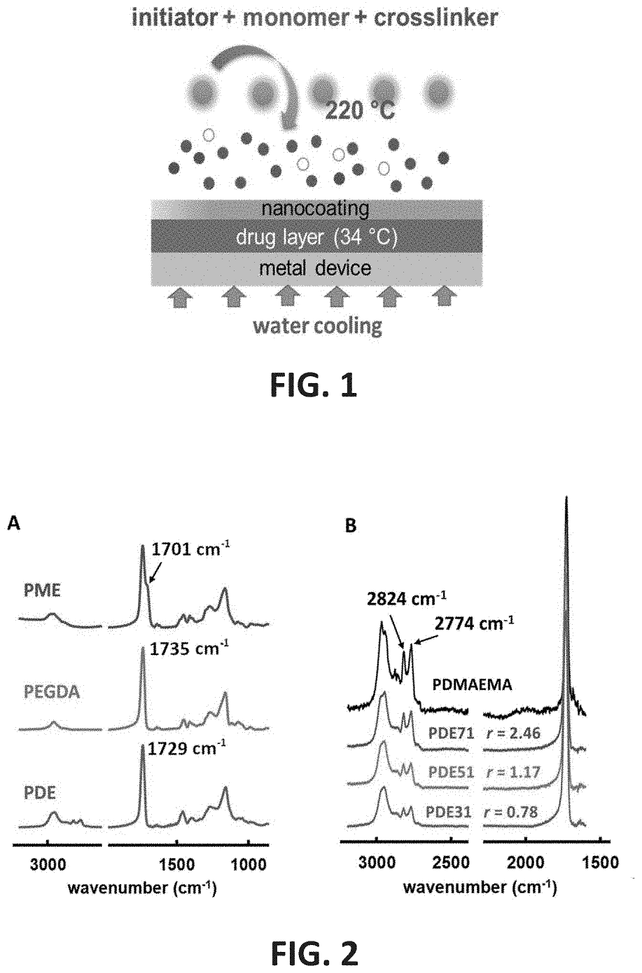

FIG. 1 schematically illustrates the vapor deposition of coatings in accordance with this Example.

Characterizations. Fourier transform infrared (FTIR) spectra of the nanocoatings were collected by a Nicolet 6700 spectrometer (Thermo Scientific, Waltham, Mass.) with a potassium bromide beamsplitter and a DTGS detector. The spectra were collected using the transmission mode at a 4 cm⁻¹ resolution from 400 to 4000 cm⁻¹. FEI Quanta 600 field-emission gun scanning electron microscope (SEM) was used to observe the morphology of the nanocoated metal stent with an acceleration voltage of 25 kV.

Measurements of Drug Release Kinetics. The nanocoated stainless steel with atorvastatin or sirolimus was placed in separate Eppendorf tubes with 1 ml of pH 7.4 phosphate-buffered saline (PBS), which were positioned in a shaker at 100 rpm and incubated at 37° C. for elution tests (Yang et al., 2010; and Stigter et al., 2002). Samples were transferred to new Eppendorf tubes with 1 ml fresh PBS each day. The collected elutes were filtered through a 0.22 μm syringe filter. Drug concentration was measured using a high-performance liquid chromatography (HPLC) system (UltiMate 3000, Thermo Fisher Scientific, Waltham, Mass.) with a C18 column (Shimadzu, Kyoto, Japan). The HPLC analysis of atorvastatin was carried out using 60% methanol and 40% deionized water as an isocratical mobile phase at a flow rate of 0.5 ml/min. The absorbency of atorvastatin was measured at the wavelength of 245 nm (Kim et al., 2008). The absorbency of sirolimus was measured at 278 nm using 80% methanol and 20% deionized water with 10 mmol/L NH4OH/HAc as an isocratical mobile phase at a flow rate of 0.8 ml/min (Ferron et al., 1997). A standard curve with concentrations ranging from 0.1 to 500 μg/ml was prepared for each set of measurement. All experiments were performed in triplicate.

Human Coronary Artery Cell Culture and Cell Activity Study. Human coronary artery smooth muscle cells (HCASMC) were purchased from Lonza (Basel, Switzerland). HCASMC was cultured with SmBM Basal Medium (Lonza) supplemented with SmGM-2 SingleQuot Kit at 37° C. with 5% CO₂ in a humidified incubator. The nanocoated atorvastatin devices were placed in 12-well plates (Greiner, Kremsmünster, Austria) for cell seeding at a density of 2500/ml. After overnight adhesion, samples were transferred to new 12-well plates to exclude cells attached to the wall of wells. Cells were cultured for a maximum of 14 days to avoid cell aggregation. Cell culture medium was replaced every other day. Samples were washed with PBS for three times before the tests of metabolism and proliferation activity. Stainless steel was used as the control in metabolism and proliferation activity tests.

Cell metabolism was examined using a 3-(4,5-dimethylthiazol-2-yl)-2,5-diphenyltetrazolium bromide (MU) assay. The cell culture devices were incubated with 500 μl fresh medium with 50 μl of MU reagent (ATCC, Manassas, Va.) in 12-well plates for three hours. After the incubation, 500 μl of SDS-HCI solution was added into each well. To fully dissolve MTT-formazan, samples were placed in dark overnight with gentle shaking. The absorbance of the supernatant of each sample was measured at 570 nm using a plate reader.

Cell proliferation activity was measured using a 5-bromo-2-deoxyuridine (BrdU) Cell Proliferation Assay Kit (BioVision, Milpitas, Calif.) following the manufacturer's protocol. The cell culture devices were incubated with 360 μl fresh medium with 40 μl of 10× BrdU solution in 12-well plates for one hour with gentle shaking. Each well was then washed with 350 μl 1× Wash Buffer for two times, incubated with 400 μl of 1× Anti-mouse HRP-linked Antibody Solution at 21° C. for 1 hour, and washed with 400 μl of 1× Wash Buffer for three times. After that, each well was incubated with 400 μl of TMB Substrate at 21° C. for 30 min. Finally, 100 μl of Stop Solution was added into each well. BrdU incorporation was determined by measuring the absorbance at 450 nm using a plate reader.

Hemocompatibility Study. Human whole blood donated by a local medical agency was centrifuged at 200 g for 10 min at 21° C. to collect the supernatant as the platelet-rich plasma (PRP). After incubation in 500 μl of normal saline at 37° C. for 30 min, the nanocoated atorvastatin devices were transferred into PRP and incubated at 37° C. for 2 h. After washing with PBS for two times, adhered platelets were fixed with 4% paraformaldehyde (Sigma-Aldrich, St. Louis, Mo.) in 0.1 M PBS for 1 h at 21° C. and incubated with 1 μg/ml of FITC anti-human CD61 (Biolegend, San Diego, Calif.) in a humidified dark box for 1 h. Platelets adhered on the sample surface were assessed under DMl3000M fluorescence microscope (Leica, Wetzlar, Germany) with the image analysis of Leica Application Suite. The number of adhered platelets were counted using image analysis of Leica Application Suite (Buffalo Grove, Ill.). Hemolysis was assessed by incubation of samples with diluted red blood cells. Samples were dipped in a microcentrifuge tube containing 1.0 ml of normal saline that were previously incubated at 37° C. for 30 min. Then 0.2 ml of 10% suspended red blood cells with normal saline was added to each sample tube, and the mixtures were incubated for 60 min at 37° C. 20% Triton X-100 (Sigma-Aldrich, St. Louis, Mo.) in deionized water was used as a positive control. After the incubation, all samples were centrifuged for 5 min at 3000 rpm, and the supernatant was carefully transferred to the cuvette for spectroscopic analysis at 545 nm. Stainless steel was used as the control in both hemolysis and platelet adhesion tests.

Statistical Analysis. The SAS software (SAS Institute, Cary, N.C.) was used for statistical analysis in this Example. The one-way analysis of variance (ANOVA, p value <0.05) was used to determine whether there were statistically significant differences between the means of three or more independent groups. Student's t-test (p value <0.05) was used afterwards to identify any statistically significant difference between two groups.

### Results and Discussion

Vapor Deposition of Nanocoatings. PME and PDE nanocoatings were deposited using MAA and DMAEMA as the monomer in the feed vapor, respectively. EGDA was used to crosslink the nanocoatings in situ. The nanocoating process started with thermal decomposition of the initiator TBP to create free radicals in the vapor phase, followed by co-polymerization of the monomer and the crosslinker, forming an encapsulation coating on the drug layer (Zhi et al., 2018). The nanocoating composition was characterized using FTIR (Panel A of FIG. 2). The absorption peaks of 1701 cm⁻¹, 1729 cm⁻¹, and 1735 cm⁻¹ were assigned to the C═O stretching of carboxyl in the MAA, DMAEMA, and EGDA moieties, respectively (Huang et al., 2002; DeSousa et al., 2010; and Chan et al. 2006), and the absorption peaks at 2774 and 2824 cm⁻¹ were assigned to the C—H stretching of tertiary amine in the DMAEMA moiety (Yliniemi et al., 2014).

Nanocoatings with different degrees of crosslinking were formed by varying the flow ratio of DMAEMA to EGDA. The peak area of tertiary amine was observed to increase with the flow ratio, indicating more DMAEMA incorporated into the composition (Panel B of FIG. 2). The molar ratio of DMAEMA to EGDA was calculated using FTIR analysis. All the spectra were normalized to the thickness of nanocoatings. The peak area ratio of the C═O stretching at 1729 cm⁻¹ (A′_{C═O}) to the N—C—H stretching at 2773 cm⁻¹ (A′_{N-C—H}) in PDMAEMA was calculated and denoted as X=A′_{C═O}/A′_{N—C—H}. The peak areas of the C═O (A_{C═O}) and N—C—H stretching (A_{N—C—H}) in PDE31, PDE51, and PDE71 were measured. The peak area of the C═O stretching contributed by DMAEMA was calculated as XA_{N—C—H}, and the area of the C═O stretching contributed by EGDA was expressed as A_{C═O}−XA_{N—C—H}. If assuming that the absorption coefficient of the C═O stretching is the same in DMAEMA and EGDA units, the molar ratio of DMAEMA to EGDA can be calculated according to the Beer-Lambert law (Ricci et al. 1994) as the ratio of C═O stretching peak area in DMAEMA to the C═O stretching peak area in EGDA:

$${r}^{′} = \frac{2 {XA}_{N - C - H}}{{A}_{C = O} - {XA}_{N - C - H}} \qquad ( 1 )$$

A factor of 2 was introduced because of two C═O bonds in each EGDA unit.

It should be noted that the absorption coefficient of the C═O stretching in DMAMEA and EGDA may vary, and the ratio (R) can be calculated using the equation in our previous study (Ye et al., 2011):

$$R = \frac{2 {A}_{C = O ( PDMAEMA )} {M}_{DMAEMA}}{{A}_{C = O ( PEGDA ) )} {M}_{EGDA}}$$

where A_{C═O(PDMAEMA)} is the C═O stretching absorbance in PDMAEMA, A_{C═O(PEGDA)} is the C═O stretching absorbance in PEGDA, and M_{EGDA} and M_{DMAEMA} are the molecular weights of EGDA and DMAEMA, respectively. The molar ratio of DMAEMA to EGDA was corrected as:

$$r = \frac{2 {XA}_{N - C - H}}{R ( {A}_{C = O} - {XA}_{N - C - H} )} \qquad ( 2 )$$

The calculated r values were listed in Panel B of FIG. 2.

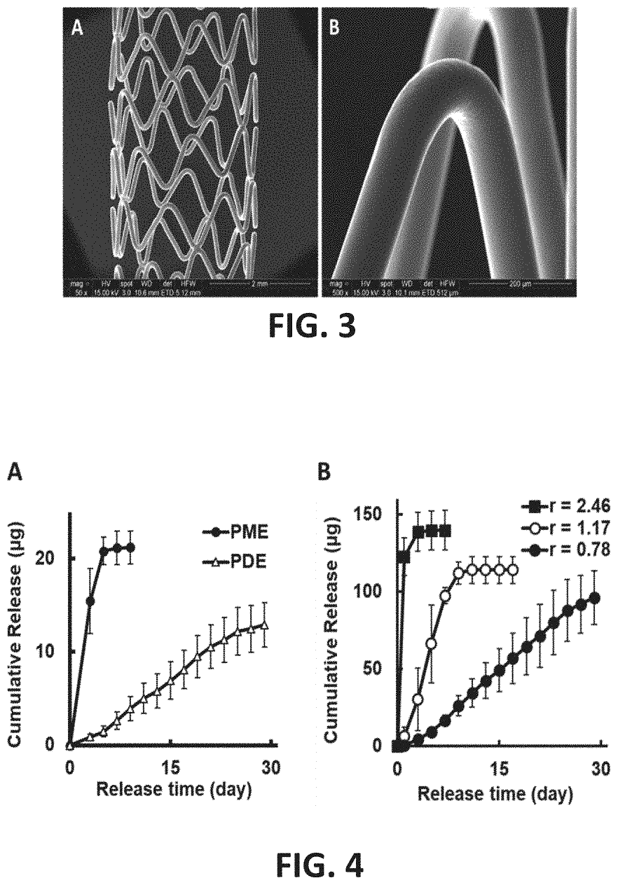

The elimination of solvent use during chemical vapor deposition (CVD) enabled fabrication of conformal coatings with excellent stability. Conventional solvent-based fabrication such as spray or dip coating results in flaking at the coating-metal interface and delamination of coatings (Hargsoon et al. 2008; and Sommakia et al., 2014), which causes obstruction of an artery and subsequent adverse cardiac events (Levy et al., 2009). On the PDE31 nanocoated stent, no delamination or cracking was observed (Panel A of FIG. 3) after delivery balloon expansion at pressure of 9 atm. Panel B of FIG. 3 shows a close view of the nanocoated stent struts, indicating that the vapor deposited nanocoating was ductile with excellent adhesion (Zhi et al. 2018; and Ye et al., 2012) with the metal underneath so as to extend with the expansion of the stent.

Drug Release Kinetics. The chemical composition of nanocoatings plays an important role in determining the release kinetics of atorvastatin. Panel A of FIG. 4 compares the 30-day release profile of PME and PDE nanocoatings. The PDE nanocoating demonstrated constant atorvastatin release rate with no burst release, while the PME nanocoating released 61.8% of encapsulated atorvastatin on the first day. Drug release through barrier coatings in aqueous medium is determined by the interplay between the coating, the drug, and water. The water contact angle of the PDE and PME nanocoatings was measured to be in the range of 61° C.-62° C., indicating a similar affinity with water. Thus, the significant difference in the release kinetics of PME and PDE nanocoatings is attributed to their difference in interacting with atorvastatin. At pH 7.4, the tertiary amine in DMAEMA becomes protonated (Rawlinson et al., 2010), resulting in positive charges in PDE, while in the PME nanocoating, the de-protonation of methacrylic acid results in negative charges (Schuwer et al., 2011). Since atorvastatin is negatively charged at pH 7.4 (pKa=4.33; Arghavani-Beydokhti et al., 2017), it is speculated that the repulsive electrostatic force in PME facilitates the mass transfer of atorvastatin through the nanocoating at such small thickness.

The atorvastatin release from PDE31, PDE51, and PDE71 was also studied (Panel B of FIG. 4). The PDE71 nanocoating with r =2.46 had burst effect with 81.6% of atorvastatin released on the first day, while the PDE51 and PDE31 nanocoatings were observed to release atorvastatin evenly. The average daily release rate was calculated to be 3.3±0.6 and 12.5±0.8 μg/day for PDE 31 (r=0.78) and PDE51 (r=1.17), respectively.

The effect of molar ratio r on drug release rate can be attributed to the change in the matrix density and the surface hydration of nanocoatings. The distance between two crosslinking points, l, was estimated to be

l=l_{DMAEMA}·r+l_{EGDA} (3)

where lEGDA is the length of the EGDA structural unit, and l_{DMAEMA} is the length of DMAEMA structural unit. Using Calculator Plugins of Marvin 18.10.0 (ChemAxon) for structure prediction, l_{EGDA} was calculated to be 0.44 nm. l_{DMAEMA} was estimated to be 0.20 nm based on the study done by Murata et al. (2013). The calculated l values are listed in Table 1. The molecular dimension of atorvastatin was estimated to be 0.57 nm (da Costa et al., 2012). As the distance l increased to render the pores inside nanocoatings significantly outsize the dimension of atorvastatin, the mass transfer tended to proceed in a diffusion controlled manner, resulting in the fast-then-slow drug release behavior (Mwangi et al., 2004). A higher molar ratio also leads to an increase in water uptake and swelling capability (Remunan Lopez et al., 1997), resulting in higher water permeability and drug diffusion rate. The increase of coating hydration at the higher molar ratio was reflected in the decrease of the water contact angle (Table 1).

TABLE 1
Distance Between Crosslinks and the
Contact Angles of Nanocoatings

| Nanocoating | r | l (nm) | CA (°) |
|---|---|---|---|
| PDE31 | 0.78 | 0.60 | 61.1 ± 0.8 |
| PDE51 | 1.17 | 0.67 | 57.1 ± 0.1 |
| PDE71 | 2.46 | 0.93 | 54.4 ± 0.4 |

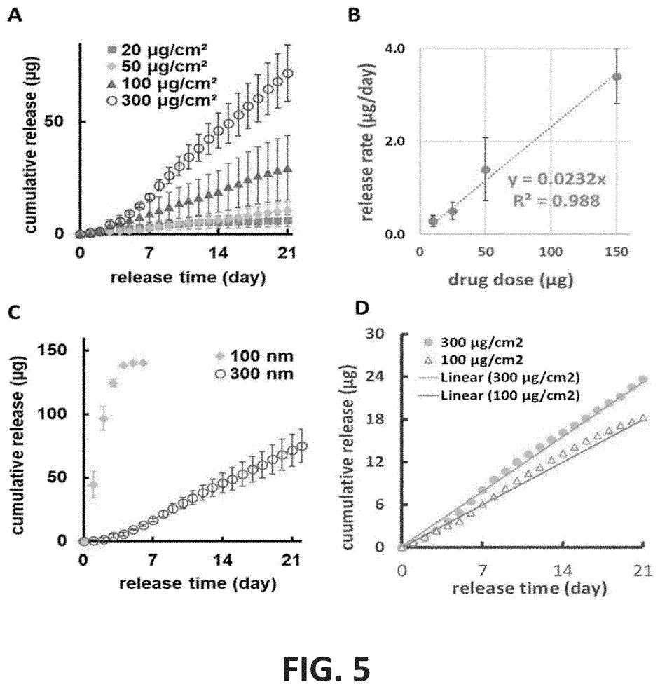

The release kinetics of nanocoated atorvastatin at varying dose density and nanocoating thickness were investigated. At the dose densities of 20, 50, 100, and 300 μg/cm², atorvastatin coated with 300 nm PDE 31 exhibited linear drug release (Panel A of FIG. 5). The release rate, calculated as Mₜ/t, where Mₜ is the mass of released atorvastatin and t is the release time, was observed to linearly increase with the accruement of drug dose, M₀. The slope of the linear regression line for daily release rate versus drug dose was calculated to be 0.0232 day⁻¹ for the 300-nm PDE31 nanocoating (Panel B of FIG. 5). This linear correlation reveals that the release rate can be modulated based on the drug dose, providing a simple but efficient approach in adjusting the drug release rate. Panel C of FIG. 5 compares the release profiles of nanocoated atorvastatin at two different thicknesses. The 100-nm coating demonstrated much faster atorvastatin release than the 300-nm coating, indicating the dominant effect of thickness on the mass transfer of drug molecules.

The release kinetics of sirolimus through PDE31 were similar to that of atorvastatin, as shown in Panel D of FIG. 5. The higher dose density of sirolimus presented a faster release rate than that of the lower dose density. The total amount of released sirolimus was observed to increase with the release time, and the linear regression gave 0.99 for the goodness of fitting, R².

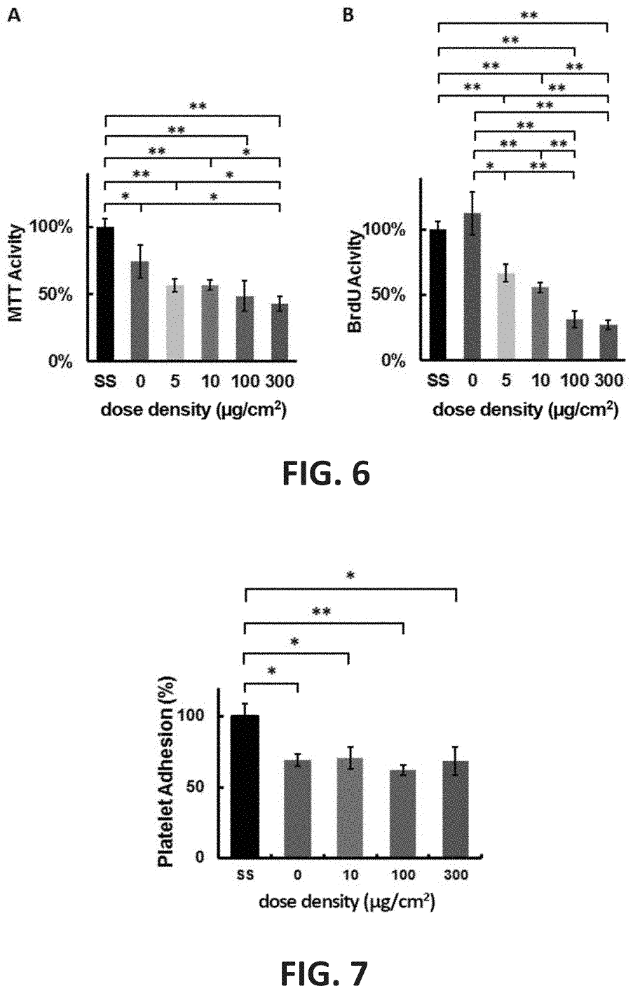

Cell Viability and Proliferation Activity. The metabolic activity of HCASMC on the PDE31 nanocoating was reduced to 74.3±12.5% of the control (Panel A of FIG. 6). The lowered cell viability was attributed to the decrease of HCASMC adhesion on the PDE31 surface, which was measured to be 74.4±12.0% of the control using the method of cell counting. With atorvastatin release, no significant difference was observed at dose densities up to 100 μg/cm². This result is consistent with the findings that 2.5 μM atorvastatin in medium does not suppress the viability of human smooth muscle cells (Dubuis et al., 2013), because the average atorvastatin concentration was calculated to be 2.42 μM, considering that the release rate was 1.40 μg/day at 100 μg/cm², and the cell culture medium was replaced at 1.0 ml every two days.

No inhibition of HCASMC proliferation was observed on the PDE31 nanocoating (Panel B of FIG. 6). With the release of atorvastatin, however, the proliferation activity of HCASMC was significantly suppressed, and the anti-proliferative effect increased with the dose density. At the dose density of 5 μg/cm², the BrdU activity of HCASMC was reduced to 66.7% of the control. Using the linear relationship between release rate and drug dose in Panel B of FIG. 5, the atorvastatin release rate was calculated to be 0.06 μg/day at the dose density of 5 μg/cm², and the average concentration of atorvastatin in medium was estimated to be 0.11 μM. Though it has been demonstrated that atorvastatin concentration as low as 0.1 μM is effective in suppressing the growth of smooth muscle cells (Petersen et al., 2013; and Baetta et al., 1997), controlled release of atorvastatin to provide such a low concentration has not been demonstrated. The capability to effectively deliver drug using such low dose density is expected to contribute to minimizing system toxicity while inhibiting HCASMC growth (Inoue et al., 2009; and Witkowski et al., 2011).

Overall, the combination of nanocoating surface and drug release substantially reduced the viability and proliferation of HCASMC on PDE31 coated atorvastatin. For example, at 10 μg/cm², the MTT and BrdU activity of HCASMC were suppressed to 56.7% and 55.7% of the control, respectively. The results were comparable to those obtained using atorvastatin at a concentration more than five times higher than the concentration resulting from the dose density of 10 μg/cm² (Petersen et al., 2013; and Dubuis et al., 2013)

Hemocompatibility Assessment. Hemolysis on PDE31 nanocoatings with and without atorvastatin was measured to be 0.54%-0.58% of the positive control. The test result was no different from that of the stainless steel control, indicating compatibility with red blood cells. On the other hand, the platelet adhesion on PDE31 was 63.6% of the control (FIG. 7). With the release of atorvastatin, the platelet adhesion remained statistically unchanged, demonstrating that atorvastatin does not affect platelet adhesion. In comparison, stent coatings of poly(butyl methacrylate) and polystyrene copolymers have similar platelet adhesion as on stainless steel (Szott et al., 2016), and poly(lactic-co-glycolic acid) shows much higher platelet adhesion than stainless steel (Uurto et al., 2004; and Otsuka et al., 2015). The reduction of platelet adhesion on the PDE31 surface indicates the potential of creating stents with improved thrombogenic profiles (Hu et al., 2002; and Byrne et al., 2015).

### Conclusions

Polyionic nanocoatings were fabricated for drug encapsulation using a one-step vapor-deposition method. The vapor-based process not only provided coating stability and conformal coverage, but also enabled a facile approach to tailor the nanocoating composition and thickness for the regulation of drug release kinetics. The PDE31 nanocoating with the DMAEMA/EGDA molar ratio of 0.78 demonstrated zero-order release for both atorvastatin and sirolimus for 30 days, and the drug release rate was linearly correlated with the dose of encapsulated drug, providing a simple but efficient way to control drug release. The combination of nanocoating surface and drug release substantially suppressed the viability and proliferation of HCASMC at atorvastatin dose densities as low as 5 μg/cm². In addition, the nanocoating surface effectively reduced platelet adhesion without a detrimental effect on red blood cells. This Example provides insights regarding biomedical implant coatings for effective delivery of therapeutics and improvement of biocompatibility.

### Example 2

In this Example, dexamethasone (DEX), an anti-inflammatory and immune-suppressing corticosteroid drug, was encapsulated using vapor-deposited polymer coatings. The release kinetics of encapsulated devices with varying parameters, including DEX dose density, coating composition, and coating thickness, were investigated. The effect of released DEX was evaluated in the inflammatory reaction of microglia. Performance of microelectrode probes before and after the CVD DEX encapsulation was compared.

### Materials and Methods

Preparation of DEX-loaded Devices. Stainless steel (SS, 316L, Online Metals) sheets were cut to the dimension of 1 cm×0.5 cm for drug release studies. Aclar films (Ted Pella) were cut to the dimension of 1 cm×1 cm for cell culture studies. These substrates were prewashed by acetone and ethanol separately in an ultrasonic bath for 30 min, followed by three rinses of deionized water and air drying. Monopolar microelectrodes (FHC) were used as received. Dexamethasone (DEX, 98%, Alfa Aesar) was dissolved in dimethylformamide (DMF, Pharmco-Aaper) at 50 mg/mL and then serially diluted. To obtain the pre-determined dose densities of 20, 50, 100, 300, 400, and 500 μg/cm², a pipette was used to drop cast 5 μl and 10 μl of the DEX solution onto SS and Aclar substrates, respectively. The SS and Aclar substrates were leveled horizontally during drug loading and solvent evaporation. The monopolar microelectrode was taped on a glass slide and immersed in 10 μl of DEX solution at 50 mg/mL. When there was no solvent visible on the surface, the DEX-loaded devices were transferred to the vacuumed chamber to prepare for coating deposition.

Drug encapsulation. Drug encapsulation was conducted using an initiated chemical vapor deposition (iCVD) process. A divinyl monomer, ethylene glycol diacrylate (EGDA, 90%, Polysciences), was used as the precursor to form the crosslinked coating poly(ethylene glycol diacrylate) (PEGDA). EGDA and the initiator Cert-butyl peroxide (TBP, 98%, Sigma Aldrich) was vaporized at 55° C. and 22° C. and metered into the vacuumed chamber at 0.25 sccm and 0.15 sccm, respectively. EGDA flow rate was controlled by a needle valve (Swagelok), while the TBP flow rate was controlled by a mass flow controller (MKS, model 1479A). n-butyl acrylate (BA, 99%, Sigma Aldrich) was used as the precursor to form the linear coating of poly(butyl acrylate) (PBA) . BA was vaporized at 40° C. and metered using a needle valve (Swagelok) at the flow rate of 0.84 sccm. The flow rate of initiator TBP was controlled at 0.42 sccm using a mass flow controller (MKS, model 1479A). The pressure in the vacuumed chamber was maintained at 0.25 torr using a throttling valve (MKS, model 253B). While a parallel array of nichrome filament (Ni80/Cr20, Good fellow) was resistively heated to 230° C. to decompose TBP to initiate the free radical polymerization, the DEX-loaded devices were placed on the water-cooled stage with temperature controlled at 32° C. Coatings at 300, 600, and 900 nm were formed by measuring the thickness change on the reference surface of silicon wafer using an interferometry system.

For comparison, PBA solution in toluene was dip coated on the DEX-loaded SS sheets to obtain the same amount of PBA coating as in the vapor-based encapsulation. The Fourier transform infrared absorption of the carbonyl peak in PBA was used to quantify the amount of PBA coating.

Characterizations. Fluorescence was observed under DMI3000M fluorescence microscope (Leica, Wetzlar, Germany). A scanning electron microscope (SEM, FEI Quanta 600) was used to visualize the microelectrodes. Impedance spectra were obtained using a Biologic SP-150 electrochemical workstation. A solution of 0.1M phosphate buffer solution was used as the electrolyte in a three-electrode cell. The impedance was determined over a frequency range of 100-100,000 Hz.

In vitro drug release tests. The DEX-loaded devices were placed in Eppendorf tubes with 1 ml phosphate-buffered saline (PBS, pH 7.4). The Eppendorf tubes were stationed in an incubator at 37° C. The devices were transferred to new Eppendorf tubes with 1 ml fresh PBS each day. 200 μl of each elute was transferred into a 96-well plate, and the DEX concentration was analyzed using a microplate reader (Synergy, BioTek) at the wavelength of 240 nm. A standard curve with concentrations ranging from 0.2-20 μg/ml was prepared for each set of measurement. The amount of release DEX on each day was summed with the released amount on previous days to obtain the cumulative release profile. All experiments were performed in triplicate.

Microglia immunohistochemistry assay. Mouse C8-B4 microglia (CRL-2540, ATCC) was cultured in Dulbecco's modified Eagle's medium (DMEM, ATCC) supplemented with 10% fetal bovine serum, 50 U/ml penicillin, and 50 μg/ml streptomycin (Life Technologies). After culturing for one week, the cells were lifted and transferred into a six-well plate at a density of 1×10⁵ cells/ml. Lipopolysaccharides (LPS, 0127:B8, Sigma-Aldrich) were added at 100 ng/ml to stimulate the microglia cells. The DEX-loaded devices at the dose densities of 20, 50, 100, and 300 μg/cm² and the DEX-free devices coated with the same thickness of PEGDA were sterilized under UV for 4 h. The devices were transferred to wells with LPS and wells that are LPS-free, resulting in four categories of conditions: (−) LPS (−) DEX; (+) LPS (+) DEX; (+) LPS (−) DEX; and (−) LPS (+) DEX. After culturing for one day and three days, microglia cells were fixed by glutaraldehyde (50%, Electron Microscopy Science) and stained by DAPI (Invitrogen) and FITC labeled FA-11 antibody (Bio-Rad). In the three-day experiment, culture media was refreshed every day, and the LPS concentration was maintained constant at 100 ng/ml. The stained microglia were observed under a DMI3000M microscope (Leica), and digital images were taken on three randomly selected areas. The number of microglia cells was assessed by DAPI staining, and the immunofluorescence intensity was quantified by analyzing the FITC intensity using Image J, which enabled the immunofluorescence intensity per cell to be calculated. The relative immunofluorescence intensity was calculated as the percentage to that of (−) LPS, (−) DEX.

Statistical Analysis. One-way Analysis of Variance (ANOVA) and the Student's T-test were conducted using the SAS software to evaluate the immunofluorescence intensity difference between groups. In all evaluations, a p-value less than 0.05 was considered as a significant difference in statistics.

### Results And Discussion

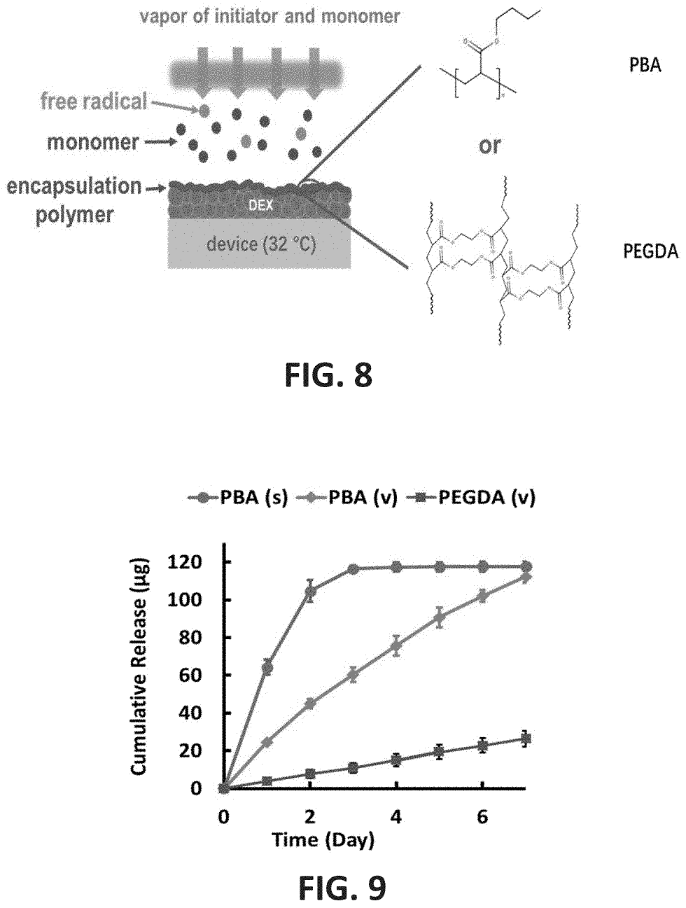

Vapor-based encapsulation of DEX. Drug encapsulation was conducted by coupling the free radical polymerization chemistry with the process of vapor deposition. The combination enabled formation of an encapsulation layer directly on drug-loaded devices. The chemical structures of linear polymer PBA and crosslinked polymer PEGDA are illustrated in FIG. 8. FIG. 9 shows the seven-day release profile of DEX encapsulated by the vapor-deposited PEGDA and PBA compared with the DEX encapsulated by solution-coated PBA. The vapor-deposited PEGDA and PBA demonstrated even delivery of DEX with no burst release, while the solution-coated PBA had over 80% of DEX released in two days. This contrast shows the efficiency and stability using the vapor-based encapsulation method.

At the encapsulation thickness of 300 nm and the dose density of 300 μg/cm², the average DEX release rate through vapor-deposited PBA and PEGDA was 16.04±4.95 μg/day and 3.760±0.45 μg/day, respectively. The significant difference in the release rate was attributed to the disparity between the linear structure in PBA and the crosslinked network in PEGDA. The formation of crosslinks caused the shrinkage of pores in the encapsulation coating, resulting in the increase of DEX mass transfer resistance and thus the reduction in release rate.

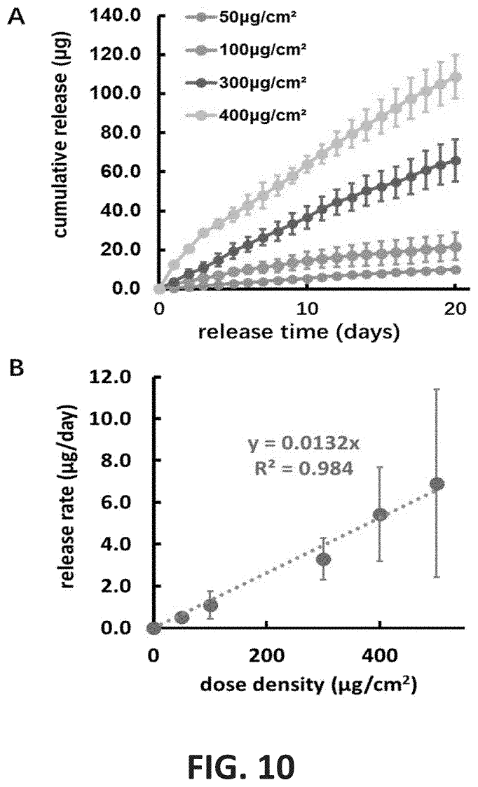

The release kinetics of PEGDA encapsulated DEX at varying dose density was studied. At the encapsulation thickness of 300 nm, the cumulative release of DEX linearly increased with time at the dose density of 50-500 μg/cm² (FIG. 10, Panel A), indicating the strong regulation of DEX release at a broad range of dose densities. The average daily release rate was linearly correlated with the DEX dose density (FIG. 10, Panel B), enabling an efficient route in modulating and predicting DEX release rate.

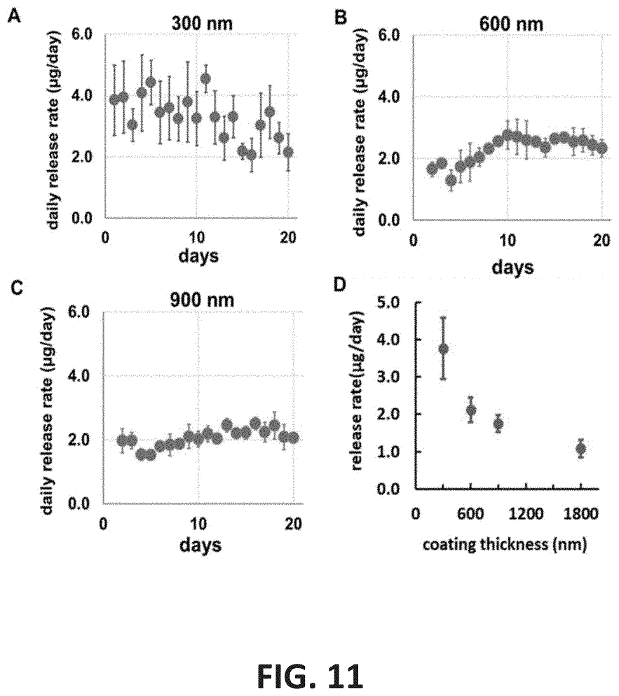

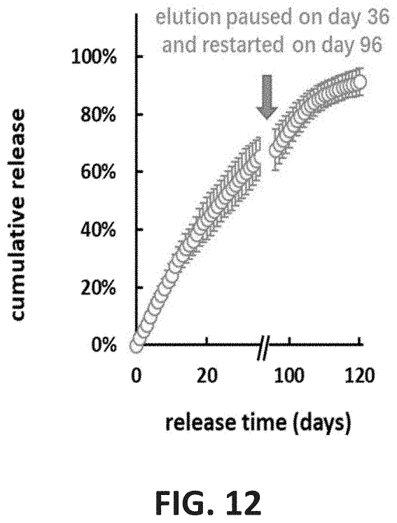

FIG. 11 compares the 20-day release profile of encapsulated DEX at three different thicknesses of PEGDA. With the increase of encapsulation thickness, the daily release rate decreased, and the fluctuation in DEX daily release significantly reduced, indicating that increasing encapsulation thickness contributes not only to the regulation of release rate but also to the stability enhancement in DEX release. The stability of PEGDA encapsulation was further confirmed in pausing DEX release on day 36 and resuming it on day 96. After storing at ambient conditions for 60 days, the DEX release kinetics remained nearly unchanged after the prolonged storage, as shown in FIG. 12.

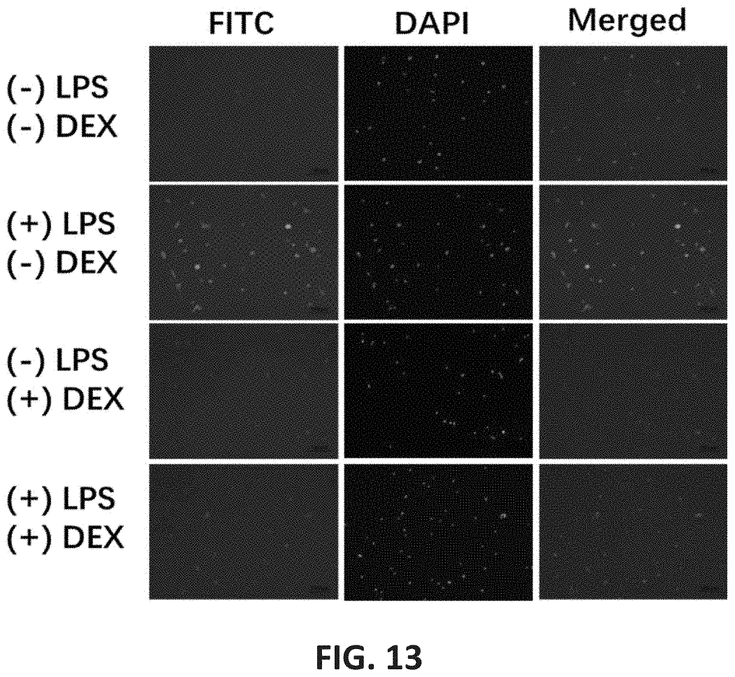

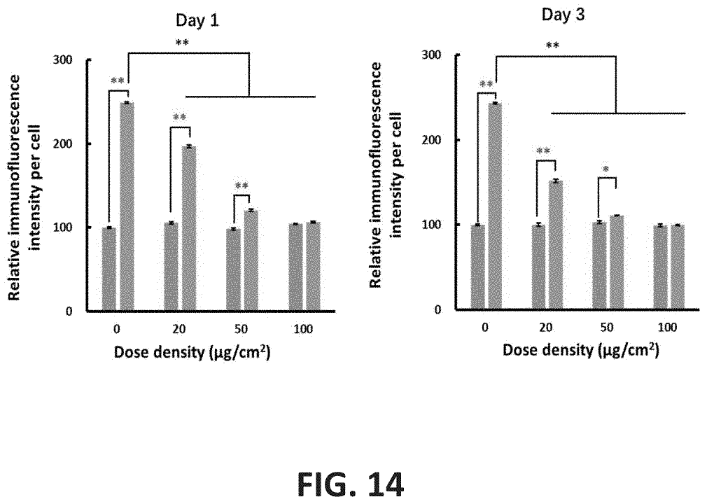

The effect of DEX released from PEGDA encapsulated devices was tested in vitro using a microglia cell study. Microglia activation after LPS stimulation was assessed via surface expression of CD68 membrane protein, which was stained by the FITC-labeled FA-11 antibody. The inhibition effect of released DEX on microglia activation was evident. As shown in FIG. 13, under LPS stimulation, the CD68 expression was substantially reduced in microglia incubated on DEX-loaded devices. After quantifying the immunofluorescence intensity per microglia cell, the relative immunofluorescence intensity per cell was calculated using microglia with no LPS stimulation and no DEX release as the control. FIG. 14 shows the results for microglia incubated on PEGDA encapsulated DEX at varying dose densities under non-stimulated and LPS-stimulated conditions. On day one, the microglia activation was suppressed by 35%, 86%, and 96% at 20, 50, and 100 μg/cm² of encapsulated DEX, respectively. On day three, the immunofluorescence intensity was further suppressed, which can be attributed to the accumulative effect of released DEX on inhibition of microglia activation. There was no significant difference between stimulated and non-stimulated microglia on PEGDA encapsulated DEX as the dose density reached 100 μg/c m².

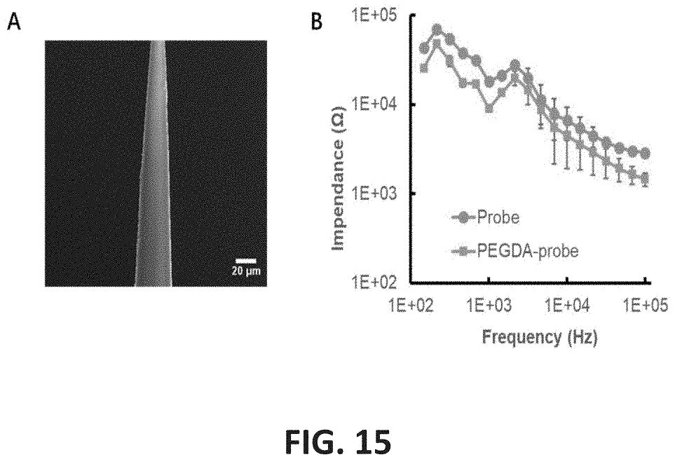

The PEGDA encapsulation of DEX had minimal effect on the morphology of the microelectrode probes. As shown in Panel A of FIG. 15, the microelectrode with DEX and PEGDA demonstrated a smooth surface without a visible difference from the pristine microelectrode. The impedance spectroscopy of the microelectrode probe showed no significant difference before and after the PEGDA encapsulation of DEX (FIG. 15, Panel B), indicating that the electrical transport between the microelectrode and the buffer solution was not hindered.

### Conclusions

DEX was encapsulated on three dimensional devices using CVD of polymer coatings at sub-micron thickness. Both linear and crosslinked polymer coatings formed by the CVD demonstrated linear delivery of DEX without burst release. The DEX release rate was linearly correlated with the dose density of DEX on devices. Increasing the encapsulation coating thickness resulted in the reduction of DEX release rate and the improvement in drug release stability. DEX released from the encapsulation device effectively suppressed the activation of microglia cells at dose density as low as 20 μg/cm². Microelectrodes showed minimal change on the morphology and the electrical transport after the vapor-based encapsulation of DEX. The CVD method represents a new means for the encapsulation of drugs and therapeutics.

While this disclosure describes the inventive concept(s) in conjunction with the specific drawings, experimentation, results, and language set forth herein, it is evident that many alternatives, modifications, and variations will be apparent to those skilled in the art. Accordingly, it is intended to embrace all such alternatives, modifications, and variations that fall within the spirit and broad scope of the present disclosure.

### REFERENCES

The following references, to the extent that they provide exemplary procedural or other details supplementary to those set forth herein, are specifically incorporated herein by reference. In addition, the following is not intended to be an Information Disclosure Statement; rather, an Information Disclosure Statement in accordance with the provisions of 37 CFR § 1.97 will be submitted separately.
Acharya, G.; Park, K. Mechanisms of controlled drug release from drug-eluting stents. Adv Drug Deliv Rev 2006, 58 (3), 387-401.
Akin et al., Second- and third-generation drug-eluting coronary stents. Herz 2011, 36(3), 190-196.
Arghavani-Beydokhti, S.; Rajabi, M.; Asghari, A. Combination of magnetic dispersive micro solid-phase extraction and supramolecular solvent-based microextraction followed by high-performance liquid chromatography for determination of trace amounts of cholesterol-lowering drugs in complicated matrices. Anal. Bioanal. Chem. 2017, 409 (18), 4395-4407.
Baetta, R.; Donetti, E.; Comparato, C.; Calore, M.; Rossi, A.; Teruzzi, C.; Paoletti, R.; Fumagalli, R.; Soma, M. R. Proapoptotic effect of atorvastatin on stimulated rabbit smooth muscle cells. Pharmacol. Res. 1997, 36 (2), 115-121.
Basalus et al., Coating irregularities of durable polymer-based drug-eluting stents as assessed by scanning electron microscopy. Eurolntervention 2009, 5 (1), 157-165.
Balakrishnan, B.; Dooley, J. F.; Kopia, G.; Edelman, E. R. Intravascular drug release kinetics dictate arterial drug deposition, retention, and distribution. J. Control. Release 2007, 123 (2), 100-8.
Bunger et al., Sirolimus-eluting biodegradable poly-L-lactide stent for peripheral vascular application: A preliminary study in porcine carotid arteries. J Surg Res 2007, 139 (1), 77-82.
Busch et al., New stent surface materials: The impact of polymer-dependent interactions of human endothelial cells, smooth muscle cells, and platelets. Acta Biomaterialia 2014, 10 (2), 688-700.

Byrne, R. A.; Joner, M.; Kastrati, A. Stent thrombosis and restenosis: what have we learned and where are we going? The Andreas Gruntzig Lecture ESC 2014. Eur. Heart J. 2015, 36 (47), 3320-+.
Cai, A. P.; Zhou, Y. L.; Li, L. W. Rho-GTPase and Atherosclerosis: Pleiotropic Effects of Statins. Journal of the American Heart Association 2015, 4 (7).
Carter, A. J.; Aggarwal, M.; Kopia, G. A.; Tio, F.; Tsao, P. S.; Kolata, R.; Yeung, A. C.; Llanos, G.; Dooley, J.; Falotico, R. Long-term effects of polymer-based, slow-release, sirolimus-eluting stents in a porcine coronary model. Cardiovasc. Res. 2004, 63 (4), 617-24.
Chan, K.; Kostun, L. E.; Tenhaeff, W. E.; Gleason, K. K. Initiated chemical vapor deposition of polyvinylpyrrolidone-based thin films. Polymer 2006, 47 (20), 6941-6947.
Chen et al., Strong resistance of phosphorylcholine self-assembled monolayers to protein adsorption: Insights into nonfouling properties of zwitterionic materials. J Am Chem Soc 2005, 127 (41), 14473-14478.
Curcio, A.; Torella, D.; Indolfi, C. Mechanisms of Smooth Muscle Cell Proliferation and Endothelial Regeneration After Vascular Injury and Stenting—Approach to Therapy. Circ. J. 2011, 75 (6), 1287-1296.
da Costa, R. F.; Freire, V. N.; Bezerra, E. M.; Cavada, B. S.; Caetano, E. W. S.; de Lima, J. L.; Albuquerque, E. L. Explaining statin inhibition effectiveness of HMG-CoA reductase by quantum biochemistry computations. Phys. Chem. Chem. Phys. 2012, 14 (4), 1389-1398.
De Sousa, F. B.; Guerreiro, J. D. T.; Ma, M.; Anderson, D. G.; Drum, C. L.; Sinisterra, R. D.; Langer, R. Photo-response behavior of electrospun nanofibers based on spiropyran-cyclodextrin modified polymer. J. Mater. Chem. 2010, 20 (44), 9910-9917.
Denardo et al., Changes to Polymer Surface of Drug-Eluting Stents During Balloon Expansion. Jama-J Am Med Assoc 2012, 307 (20), 2148-2150.
Dubuis, C.; May, L.; Alonso, F.; Luca, L.; Mylonaki, I.; Meda, P.; Delie, F.; Jordan, O.; Deglise, S.; Corpataux, J. M.; Saucy, F.; Haefliger, J. A. Atorvastatin-Loaded Hydrogel Affects the Smooth Muscle Cells of Human Veins. J. Pharmacol. Exp. Ther. 2013, 347 (3), 574-581.
Ferron, G. M.; Conway, W. D.; Jusko, W. J. Lipophilic benzamide and anilide derivatives as high-performance liquid chromatography internal standards: application to sirolimus (rapamycin) determination. J. Chromatogr. B Biomed. Sci. Appl. 1997, 703 (1-2), 243-51.
Finkelstein, A.; McClean, D.; Kar, S.; Takizawa, K.; Varghese, K.; Baek, N.; Park, K.; Fishbein, M. C.; Makkar, R.; Litvack, F.; Eigler, N. L. Local drug delivery via a coronary stent with programmable release pharmacokinetics. Circulation 2003, 107 (5), 777-84.
Fuster, V.; Kelly, B. B.; Vedanthan, R. Promoting global cardiovascular health: moving forward. Circulation 2011, 123 (15), 1671-8.
Hargsoon, Y.; Devesh, C. D.; Vasuda, R.; Vijay, K. V. Aligned nanowire growth using lithography-assisted bonding of a polycarbonate template for neural probe electrodes. Nanotechnology 2008, 19 (2), 025304.
Haude, M.; Lee, S. W.; Worthley, S. G.; Silber, S.; Verheye, S.; Erbs, S.; Rosli, M. A.; Botelho, R.; Meredith, I.; Sim, K. H.; Stella, P. R.; Tan, H. C.; Whitbourn, R.; Thambar, S.; Abizaid, A.; Koh, T. H.; Den Heijer, P.; Parise, H.; Cristea, E.; Maehara, A.; Mehran, R. The REMEDEE trial: a randomized comparison of a combination sirolimus-eluting endothelial progenitor cell capture stent with a paclitaxel-eluting stent. JACC Cardiovasc. Interv. 2013, 6 (4), 334-43.
Hu, W. J.; Eaton, J. W.; Ugarova, T. P.; Tang, L. P. Molecular basis of biomaterial-mediated foreign body reactions (vol 98, pg 1231, 2001). Blood 2002, 99 (11), 3908-3908.
Huang, Q. R.; Volksen, W.; Huang, E.; Toney, M.; Frank, C. W.; Miller, R. D. Structure and interaction of organic/inorganic hybrid nanocomposites for microelectronic applications. 1. MSSQ/P(MMA-co-DMAEMA) nanocomposites. Chem. Mater. 2002, 14 (9), 3676-3685.
Huang, Y.; Venkatraman, S. S.; Boey, F. Y.; Lahti, E. M.; Umashankar, P. R.; Mohanty, M.; Arumugam, S.; Khanolkar, L.; Vaishnav, S. In vitro and in vivo performance of a dual drug-eluting stent (DDES). Biomaterials 2010, 31 (15), 4382-91.
Hussner, J.; Begunk, R.; Boettcher, K.; Gliesche, D. G.; Prestin, K.; zu Schwabedissen, H. E. M. Expression of OATP2B1 as determinant of drug effects in the microcompartment of the coronary artery. Vascul. Pharmacol. 2015, 72, 25-34.
Inoue, M.; Sasaki, M.; Katada, Y.; Fujiu, K.; Manabe, I.; Nagai, R.; Taguchi, T. Poly-(L-lactic acid) and citric acid-crosslinked gelatin composite matrices as a drug-eluting stent coating material with endothelialization, antithrombogenic, and drug release properties. Journal of Biomedical Materials Research Part A 2013, 101 (7), 2049-2057.
Inoue, T.; Node, K. Molecular Basis of Restenosis and Novel Issues of Drug-Eluting Stents. Circ. J. 2009, 73 (4), 615-621.
Jeanmart, H.; Malo, O.; Carrier, M.; Nickner, C.; Desjardins, N.; Perrault, L. P. Comparative study of cyclosporine and tacrolimus vs newer immunosuppressants mycophenolate mofetil and rapamycin on coronary endothelial function. J. Heart Lung Transplant. 2002, 21 (9), 990-8.
Kazi et al., Association of Spontaneous Bleeding and Myocardial Infarction With Long-Term Mortality After Percutaneous Coronary Intervention. J. Am. Coll. Cardiol. 2015, 65 (14), 1411-1420.
Kim, M. S.; Jin, S. J.; Kim, J. S.; Park, H. J.; Song, H. S.; Neubert, R. H. H.; Hwang, S. J. Preparation, characterization and in vivo evaluation of amorphous atorvastatin calcium nanoparticles using supercritical antisolvent (SAS) process. Eur. J. Pharm. Biopharm. 2008, 69 (2), 454-465.
Levy, Y.; Mandler, D.; Weinberger, J.; Domb, A. J. Evaluation of drug-eluting stents' coating durability—clinical and regulatory implications. J. Biomed. Mater. Res. B Appl. Biomater. 2009, 91 (1), 441-51.
Liu, H. T.; Li, F.; Wang, W. Y.; Li, X. J.; Liu, Y. M.; Wang, R. A.; Guo, W. Y.; Wang, H. C. Rapamycin inhibits re-endothelialization after percutaneous coronary intervention by impeding the proliferation and migration of endothelial cells and inducing apoptosis of endothelial progenitor cells. Tex. Heart Inst. J. 2010, 37 (2), 194-201.
Martinez, A. W.; Caves, J. M.; Ravi, S.; Li, W. S.; Chaikof, E. L. Effects of crosslinking on the mechanical properties, drug release and cytocompatibility of protein polymers. Acta Biomater. 2014, 10 (1), 26-33.
Mozaffarian, D.; Benjamin, E. J.; Go, A. S.; Arnett, D. K.; Blaha, M. J.; Cushman, M.; Das, S. R.; de Ferranti, S.; Despres, J. P.; Fullerton, H. J.; Howard, V. J.; Huffman, M. D.; Isasi, C. R.; Jimenez, M. C.; Judd, S. E.; Kissela, B. M.; Lichtman, J. H.; Lisabeth, L. D.; Liu, S.; Mackey, R. H.; Magid, D. J.; McGuire, D. K.; Mohler, E. R., 3rd; Moy, C. S.; Muntner, P.; Mussolino, M. E.; Nasir, K.; Neumar, R. W.; Nichol, G.; Palaniappan, L.; Pandey, D. K.; Reeves, M. J.; Rodriguez, C. J.; Rosamond, W.; Sorlie, P. D.; Stein, J.; Towfighi, A.; Turan, T. N.; Virani, S. S.; Woo, D.; Yeh, R. W.; Turner, M. B.; American Heart Association Statistics, C.; Stroke Statistics, S. Executive Summary: Heart Disease and Stroke Statistics—2016 Update: A Report From the American Heart Association. Circulation 2016, 133 (4), 447-54.
Murata, H.; Cummings, C. S.; Koepsel, R. R.; Russell, A. J. Polymer-Based Protein Engineering Can Rationally Tune Enzyme Activity, pH-Dependence, and Stability. Biomacromolecules 2013, 14 (6), 1919-1926.
Mwangi, J. W.; Ofner, C. M., 3rd. Crosslinked gelatin matrices: release of a random coil macromolecular solute. Int. J. Pharm. 2004, 278 (2), 319-27.
Otsuka, F.; Cheng, Q.; Yahagi, K.; Acampado, E.; Sheehy, A.; Yazdani, S. K.; Sakakura, K.; Euller, K.; Perkins, L. E. L.; Kolodgie, F. D. Acute Thrombogenicity of a Durable Polymer Everolimus-Eluting Stent Relative to Contemporary Drug-Eluting Stents With Biodegradable Polymer Coatings Assessed Ex Vivo in a Swine Shunt Model. JACC Cardiovasc. Interv. 2015, 8 (9), 1248-1260.
Pendyala, L. K.; Li, J.; Shinke, T.; Geva, S.; Yin, X.; Chen, J. P.; King, S. B., 3rd; Robinson, K. A.; Chronos, N. A.; Hou, D. Endothelium-dependent vasomotor dysfunction in pig coronary arteries with Paclitaxel-eluting stents is associated with inflammation and oxidative stress. JACC Cardiovasc. Interv. 2009, 2 (3), 253-62.
Petersen, S.; Hussner, J.; Reske, T.; Grabow, N.; Senz, V.; Begunk, R.; Arbeiter, D.; Kroemer, H. K.; Schmitz, K. P.; Schwabedissen, H. E. M. Z.; Sternberg, K. In vitro study of dual drug-eluting stents with locally focused sirolimus and atorvastatin release. Journal of Materials Science-Materials in Medicine 2013, 24 (11), 2589-2600.
Rawlinson, L. A. B.; Ryan, S. M.; Mantovani, G.; Syrett, J. A.; Haddleton, D. M.; Brayden, D. J. Antibacterial Effects of Poly(2-(dimethylamino ethyl)methacrylate) against Selected Gram-Positive and Gram-Negative Bacteria. Biomacromolecules 2010, 11 (2), 443-453.
RemunanLopez, C.; Bodmeier, R. Mechanical, water uptake and permeability properties of crosslinked chitosan glutamate and alginate films. J. Control. Release 1997, 44 (2-3), 215-225.
Ricci, R. W.; Ditzler, M.; Nestor, L. P. Discovering the Beer-Lambert Law. J. Chem. Educ. 1994, 71(11), 983.
Ritger, P. L.; Peppas, N. A. A simple equation for description of solute release I. Fickian and non-fickian release from non-swellable devices in the form of slabs, spheres, cylinders or discs. J. Control. Release 1987, 5 (1), 23-36.
Ritger, P. L.; Peppas, N. A. A simple equation for description of solute release II. Fickian and anomalous release from swellable devices. J. Control. Release 1987, 5 (1), 37-42.
Ruggeri et al. Adhesion mechanisms in platelet function. Circ Res 2007, 100 (12), 1673-1685.
Schuwer, N.; Klok, H. A. Tuning the pH Sensitivity of Poly(methacrylic acid) Brushes. Langmuir 2011, 27 (8), 4789-4796.
Serruys, P. W.; Sianos, G.; Abizaid, A.; Aoki, J.; den Heijer, P.; Bonnier, H.; Smits, P.; McClean, D.; Verheye, S.; Belardi, J.; Condado, J.; Pieper, M.; Garnbone, L.; Bressers, M.; Symons, J.; Sousa, E.; Litvack, F. The effect of variable dose and release kinetics on neointimal hyperplasia using a novel paclitaxel-eluting stent platform—The paclitaxel in-stent controlled elution study (PISCES). J. Am. Coll. Cardiol. 2005, 46 (2), 253-260.
Sommakia, S.; Lee, H. C.; Gaire, J.; Otto, K. J. Materials approaches for modulating neural tissue responses to implanted microelectrodes through mechanical and biochemical means. Curr. Opin. Solid State Mater. Sci. 2014, 18 (6), 319-328.
Sousa, J. E.; Costa, M. A.; Abizaid, A.; Feres, F.; Seixas, A. C.; Tanajura, L. F.; Mattos, L. A.; Falotico, R.; Jaeger, J.; Popma, J. J.; Serruys, P. W.; Sousa, A. G. Four-year angiographic and intravascular ultrasound follow-up of patients treated with sirolimus-eluting stents. Circulation 2005, 111 (18), 2326-9.
Stettler, C.; Wandel, S.; Allemann, S.; Kastrati, A.; Morice, M. C.; Schomig, A.; Pfisterer, M. E.; Stone, G. W.; Leon, M. B.; de Lezo, J. S.; Goy, J. J.; Park, S. J.; Sabate, M.; Suttorp, M. J.; Kelbaek, H.; Spaulding, C.; Menichelli, M.; Vermeersch, P.; Dirksen, M. T.; Cervinka, P.; Petronio, A. S.; Nordmann, A. J.; Diem, P.; Meier, B.; Zwahlen, M.; Reichenbach, S.; Trelle, S.; Windecker, S.; Juni, P. Outcomes associated with drug-eluting and bare-metal stents: a collaborative network meta-analysis. Lancet 2007, 370 (9591), 937-48.
Stigter, M.; de Groot, K.; Layrolle, P. Incorporation of tobramycin into biomimetic hydroxyapatite coating on titanium. Biomaterials 2002, 23 (20), 4143-4153.
Sun, D.; Zheng, Y.; Yin, T.; Tang, C.; Yu, Q.; Wang, G. Coronary drug-eluting stents: from design optimization to newer strategies. J. Biomed. Mater. Res. A 2014, 102 (5), 1625-40.
Szott, L. M.; Irvin, C. A.; Trollsas, M.; Hossainy, S.; Ratner, B. D. Blood compatibility assessment of polymers used in drug eluting stent coatings. Biointerphases 2016, 11 (2), 029806.
Uurto, I.; Juuti, H.; Parkkinen, J.; Kellomäki, M.; Keskinisula, L.; Nevalainen, T.; Törmälä, P.; Salenius, J. P. Biodegradable self-expanding poly-L/D-lactic acid vascular stent: a pilot study in canine and porcine iliac arteries. Journal of Endovascular Therapy An Official Journal of the International Society of Endovascular Specialists 2004, 11 (6), 712.
Venkatraman, S.; Boey, F. Release profiles in drug-eluting stents: issues and uncertainties. J. Control. Release 2007, 120 (3), 149-60.
Wessely, R., New drug-eluting stent concepts. Nature Reviews Cardiology 2010, 7(4), 194-203.
Witkowski, A.; Opolski, M. P.; Dudek, D.; Lesiak, M.; Legutko, J.; Wojakowski, W.; Ochala, A.; Maciejewski, P.; Deptuch, T.; Araszkiewicz, A.; Ciecwierz, D.; Gil, R.; Grajek, S.; Polonski, L.; Kardiologicz, P. T. Current status of drug-eluting stents and drug-eluting balloons in patients with stable coronary artery disease: an expert consensus document of the Association for Percutaneous Cardiovascular Interventions and the Polish Cardiac Society. Postepy W Kardiologii Interwencyjnej 2011, 7 (1), 20-55.
Xu, X. H.; Lu, P.; Guo, M. Q.; Fang, M. Z. Cross-linked gelatin/nanoparticles composite coating on micro-arc oxidation film for corrosion and drug release. Appl. Surf. Sci. 2010, 256 (8), 2367-2371.
Yang, Y. J.; Tao, X.; Hou, Q.; Ma, Y.; Chen, X. L.; Chen, J. F. Mesoporous silica nanotubes coated with multilayered polyelectrolytes for pH-controlled drug release. Acta Biomater. 2010, 6 (8), 3092-100.
Ye, Y. M.; Mao, Y. Vapor-based synthesis of ultrathin hydrogel coatings for thermo-responsive nanovalves. J. Mater. Chem. 2011, 21 (22), 7946-7952.
Ye et al., Single-step fabrication of non-leaching antibacterial surfaces using vapor crosslinking. J. Mater. Chem. 2011, 21 (1), 257-262.
Ye, Y. M.; Mao, Y.; Wang, H. Z.; Ren, Z. F. Hybrid structure of pH-responsive hydrogel and carbon nanotube array with superwettability. J. Mater. Chem. 2012, 22 (6), 2449-2455.
Ye, Y.; Mao, Y. Vapor-based synthesis and micropatterning of Janus thin films with distinct surface wettability and mechanical robustness. Rsc Advances 2017, 7 (40), 24569-24575.
Ye, Y.; Mao, Y.; Wang, H. Z.; Ren, Z. F. Hybrid structure of pH-responsive hydrogel and carbon nanotube array with superwettability. J. Mater. Chem. 2012, 22 (6), 2449-2455.
Ye, Y.; Song, Q.; Mao, Y. Solventless hybrid grafting of antimicrobial polymers for self-sterilizing surfaces. J. Mater. Chem. 2011, 21 (35), 13188-13194.
Yliniemi, K.; Wilson, B. P.; Singer, F.; Höhn, S.; Kontturi, E.; Virtanen, S. Dissolution Control of Mg by Cellulose Acetate—Polyelectrolyte Membranes. ACS Applied Materials & Interfaces 2014, 6 (24), 22393-22399.
Zaverio M, R.; G Loredana, M. Adhesion mechanisms in platelet function. Circ. Res. 2007, 100 (12), 1673.
Zhi, B.; Song, Q.; Mao, Y. Vapor deposition of polyionic nanocoatings for reduction of microglia adhesion. Rsc Advances 2018, 8 (9), 4779-4785.

What is claimed is:

1. A method of producing an assembly comprising a drug-eluting biomedical implant, the method comprising the steps of:
disposing at least one drug on at least a portion of at least one surface of a biomedical implant to form at least one drug layer on the biomedical implant; and
depositing at least one polymer coating on the drug layer of the biomedical implant by a solvent-free, chemical vapor deposition process; and
wherein the at least one drug is released via a controlled manner from the polymer coating.

2. The method of claim 1, wherein the chemical vapor deposition process occurs at a temperature in a range of from about 5° C. to about 70° C.

3. The method of claim 1, wherein the biomedical implant is selected from the group consisting of an arterial stent, a neural probe, an orthopedic device, a vascular prosthetic device, an endoprosthetic device, a cardiac pacemaker, an implanted cardiac defibrillator, an intraocular lens, an intrauterine device, a breast implant, a tympanostomy tube, and combinations thereof.

4. The method of claim 1, wherein the at least one drug is selected from the group consisting of an anti-proliferative drug, an anti-inflammatory drug, an anti-microbial drug, a cancer therapy drug, a protein therapeutic, a hormone, and combinations thereof.

5. The method of claim 4, wherein the at least one drug is selected from the group consisting of sirolimus, everolimus, zotarolimus, umirolimus, novolimus, amphilimus, atorvastatin, paclitaxel, dexamethasone, and combinations thereof.

6. The method of claim 1, wherein the polymer coating is formed of one or more polymers, wherein at least one of the polymers is selected from the group consisting of poly(2-dimethylamino ethyl methacrylate-co-ethylene glycol diacrylate) (PDE), poly(methacrylic acid-co-ethylene glycol diacrylate) (PME), poly(ethylene glycol dimethacrylate) (PEGDA), poly(2-dimethylamino ethyl methacrylate-co-mathacrylic acid-co-ethylene glycol dimethacrylate) (PDME), poly(butyl acrylate) (PBA), poly(1H,1H,2H,2H-perfluorodecyl acrylate) (PPFDA), poly(divinylbenzene) (PDVB), polytetrafluoroethylene (PTFE), and combinations thereof.

7. The method of claim 6, wherein the polymer coating comprises PDE, and wherein the at least one drug comprises atorvastatin.

8. The method of claim 6, wherein the polymer coating comprises PBA or PEGDA, and wherein the at least one drug comprises dexamethasone.

9. The method of claim 1, wherein the polymer coating has a thickness in a range of from about 100 nm to about 10 micron.

10. The method of claim 1, wherein the polymer coating has a thickness in a range of from about 200 nm to about 1.0 micron.

11. The method of claim 1, wherein the at least one drug is present in the polymer coating in a concentration in a range of from about 10 μg/cm² to about 500 mg/cm².

12. The method of claim 1, wherein the assembly has an average daily drug release rate in a range of from about 0.1 μg/day to about 5 mg/day.

13. The method of claim 1, wherein the assembly exhibits controlled drug release for a period in a range of from about one week to about 50 weeks.

14. The method of claim 1, wherein the assembly exhibits a close-to-linear release of drug with substantially no burst release of drug.

15. The method of claim 1, wherein the solvent-free, chemical vapor deposition process is a single-step, solvent-free, chemical vapor deposition process.

16. The method of claim 1, wherein the assembly is substantially biocompatible with at least one of human endothelial cells, vascular smooth muscle cells, and platelets.

17. An assembly produced by the method of claim 1, the assembly comprising:
a biomedical implant;
a polymer coating deposited on at least a portion of at least one surface of the biomedical implant via a solvent-free, chemical vapor deposition process; and
at least one drug disposed within the coating; and
wherein the assembly exhibits controlled release of the at least one drug via elution from the polymer coating.

18. A kit, comprising:
at least one assembly of claim 17.

19. A method, comprising the step of:
placing an assembly within a patient in need thereof, wherein the assembly comprises a biomedical implant having a polymer coating deposited on at least a portion of at least one surface of the biomedical implant via a solvent-free, chemical vapor deposition process, the assembly further comprising at least one drug disposed within the coating; and
wherein the assembly exhibits controlled release of the at least one drug via elution from the polymer coating within the patient.

20. The method of claim 19, wherein the biomedical implant of the assembly is an arterial stent, and wherein the placing step is further defined as placing the assembly within an artery of the patient.
